(19) United States
(12) Patent Application Publication
(10) Pub. No.: US 2026/0157731 A1
(43) Pub. Date: Jun. 11, 2026

(54) METHOD OF PROVIDING INFORMATION ABOUT ELASTOGRAPHY AND ULTRASOUND IMAGING APPARATUS THEREFOR

(71) Applicant: Samsung Medison Co., Ltd., Hongcheon-gun (KR)

(72) Inventors: Chulhee YUN, Seoul (KR); Donggeon KONG, Seoul (KR)

(21) Appl. No.: 19/215,458

(22) Filed: May 22, 2025

(30) Foreign Application Priority Data
Dec. 6, 2024 (KR) 10-2024-0180227

(51) Int. Cl. A61B 8/00; G06V 10/25

(52) U.S. Cl. CPC A61B 8/485; A61B 8/5215; G06V 10/25

(57) ABSTRACT

An ultrasound imaging apparatus includes an ultrasound transceiver module, a memory storing instructions, and at least one processor including processing circuitry, wherein, when the instructions are executed individually or collectively by the at least one processor, the ultrasound imaging apparatus is configured to repeatedly perform elastography on a region of interest (ROI) through the ultrasound transceiver module, based on receiving a user input for obtaining an elasticity value of the ROI of an object, identify an elasticity measurement ROI on an elasticity image when the elasticity image of the ROI is obtained through the elastography, obtain an elasticity value from the identified elasticity measurement ROI, accumulate the obtained elasticity value as elasticity data, display information about the number of the accumulated elasticity data together with the obtained elasticity image, and obtain a next elasticity image through next elastography.

### CROSS-REFERENCE TO RELATED APPLICATION

This application is based on and claims priority under 35 U.S.C. § 119 to Korean Patent Application No. 10-2024-0180227, filed on Dec. 6, 2024, in the Korean Intellectual Property Office, the disclosure of which is incorporated by reference herein in its entirety.

### BACKGROUND

### 1. Field

The disclosure relates to an ultrasound imaging apparatus providing information about elastography, a method of providing the information about elastography, and a computer-readable recording medium having stored therein a computer program to perform the method.

### 2. Description of the Related Art

Recently, in the medical field, many different types of medical imaging apparatuses are widely used to obtain information about biological tissues of the human body by imaging the information for the purpose of early diagnosis of or operations for various diseases. As representative examples of these imaging apparatuses, there may be an ultrasound imaging apparatus, a computed tomography (CT) apparatus, and a magnetic resonance imaging (MRI) apparatus.

The ultrasound imaging apparatus is an apparatus non-invasively obtaining at least one image of an internal portion (e.g., a soft tissue or a blood flow) of an object by irradiating an ultrasound signal produced from a transducer of a probe to the object and receiving information of a signal reflecting from the object. The ultrasound imaging apparatus may be used for medical purposes such as observing inside of an object, detecting foreign substances, measuring injuries, etc. Such an ultrasound imaging apparatus is widely used along with other imaging devices due to having high stability, enabling images to be displayed in real time and being free of radiation exposure thereby being safe, as compared to an X-ray based imaging apparatus.

### SUMMARY

Additional aspects will be set forth in part in the description which follows and, in part, will be apparent from the description, or may be learned by practice of the presented embodiments of the disclosure.

According to an embodiment, an ultrasound imaging apparatus is provided. The ultrasound imaging apparatus includes an ultrasound transceiver module, a memory storing instructions, and at least one processor including processing circuitry. When the instructions are executed individually or collectively by the at least one processor, the ultrasound imaging apparatus may repeatedly perform elastography on a region of interest (ROI) through the ultrasound transceiver module, based on receiving a user input for obtaining an elasticity value of the ROI of an object, identify an elasticity measurement ROI on an elasticity image when the elasticity image of the ROI is obtained through the elastography, obtain an elasticity value from the identified elasticity measurement ROI, accumulate the obtained elasticity value as elasticity data, display information about the number of the accumulated elasticity data together with the obtained elasticity image, and obtain a next elasticity image through next elastography.

According to an embodiment, a method of providing information about elastography is provided. The method includes repeatedly performing elastography on an ROI, based on receiving a user input for obtaining an elasticity value of the ROI of an object, identifying an elasticity measurement ROI on an elasticity image when the elasticity image of the ROI is obtained through the elastography, obtain an elasticity value from the identified elasticity measurement ROI, accumulating the obtained elasticity value as elasticity data, displaying information about the number of the accumulated elasticity data together with the obtained elasticity image, and obtaining a next elasticity image through next elastography.

According to an embodiment, provided is a computer-readable recording medium having recorded thereon a program that, when executed by a computer, performs the method of providing the information about elastography.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a block diagram of a configuration of an ultrasound imaging system when a probe is a wired probe or a hybrid probe;

FIG. 1B is a control block diagram of a configuration of the ultrasound imaging system when the probe is a wireless probe or a hybrid probe;

FIGS. 2A to 2D are diagrams illustrating ultrasound imaging apparatuses, according to an embodiment;

FIG. 3 illustrates a method, performed by an ultrasound imaging apparatus, of determining an elasticity value of a region of interest (ROI), according to an embodiment;

FIG. 4 illustrates a method, performed by an ultrasound imaging apparatus, of providing information about a progress of measurement of an elasticity value, according to an embodiment;

FIG. 5 is a flowchart of a method, performed by an ultrasound imaging apparatus, of measuring an elasticity value, according to an embodiment;

FIG. 6 illustrates a method, performed by an ultrasound imaging apparatus, of additionally obtaining elasticity data based on a volatility indicator of elasticity data, according to an embodiment;

FIG. 7 illustrates a method, performed by an ultrasound imaging apparatus, of determining a representative value of elasticity data by excluding outliers, according to an embodiment;

FIG. 8 illustrates a method, performed by an ultrasound imaging apparatus, of selecting a target number of elasticity values from elasticity data, according to an embodiment;

FIG. 9 is a flowchart of a method, performed by an ultrasound imaging apparatus, of displaying a progress of measurement of an elasticity value, according to an embodiment;

FIG. 10 illustrates a method, performed by an ultrasound imaging apparatus, of updating a progress of measurement of an elasticity value, according to an embodiment;

FIG. 11 illustrates a method, performed by an ultrasound imaging apparatus, of determining a progress of measurement of an elasticity value based on a user input excluding elasticity images, according to an embodiment;

FIG. 12 is a flowchart of a method, performed by an ultrasound imaging apparatus, displaying an elasticity measurement ROI on an elasticity image when the ultrasound imaging apparatus obtains the elasticity image, according to an embodiment;

FIG. 13 illustrates a method, performed by an ultrasound imaging apparatus, of determining an elasticity measurement ROI, according to an embodiment;

FIG. 14 illustrates a method, performed by an ultrasound imaging apparatus, of changing an elasticity measurement ROI based on a user input, according to an embodiment;

FIG. 15 is a flowchart of a method, performed by an ultrasound imaging apparatus, of stopping elastography based on a progress of measurement of an elasticity value, according to an embodiment; and

FIG. 16 illustrates a method, performed by an ultrasound imaging apparatus, of displaying elasticity images, according to an embodiment.

### DETAILED DESCRIPTION

Throughout the disclosure, the expression “at least one of a, b or c” indicates only a, only b, only c, both a and b, both a and c, both b and c, all of a, b, and c, or variations thereof.

Embodiments will be described more fully hereinafter with reference to the accompanying drawings so that they may be easily implemented by one of ordinary skill in the art to which the disclosure belongs. However, the disclosure may be implemented in different forms and should not be construed as being limited to the embodiments set forth herein. In addition, parts not related to descriptions of the disclosure are omitted to clearly explain the disclosure in the drawings, and like reference numerals denote like elements throughout.

As the terms used herein, general terms that are currently widely used are selected by taking functions in the disclosure into account, but the terms are intended to encompass various other terms depending on an intention of those skilled in the art, precedent cases, advent of new technologies, etc. Thus, the terms used herein should be defined not by simple appellations thereof but based on the meaning of the terms together with the overall description of the disclosure.

In addition, although the terms including an ordinal number such as “first”, “second”, etc. may be used herein to describe various elements or components, these elements or components should not be limited by the terms. The terms are only used to distinguish one element or component from another element or component.

In addition, the terms used herein are only used to describe particular embodiments, and are not intended to limit the disclosure. Singular expressions used herein are intended to include plural expressions as well unless the context clearly indicates otherwise. In addition, throughout the specification, it will be understood that when a part is referred to as being “connected” or “coupled” to another part, it may be directly connected to or electrically coupled to the other part with one or more intervening elements therebetween. In addition, throughout the specification, when a part “includes” or “comprises” an element, unless there is a particular description contrary thereto, it is understood that the part may further include other elements, not excluding the other elements.

Not all elements of embodiments of the disclosure will be described, and description of what are commonly known in the art or what overlap each other in the embodiments will be omitted. Throughout the specification, the term ‘module’ or ‘unit’ may refer to one implemented by at least one combination of software, hardware or firmware, and a plurality of modules or units may be implemented in one element or a single module or unit may include a plurality of elements depending on the embodiments.

Expressions such as “in some embodiments of the disclosure” or “in an embodiment” described in various parts of this specification do not necessarily refer to the same embodiment(s).

Also, as used herein, an ‘object’ is a target to be imaged, and may include a human, an animal, or a part thereof. For example, the object may include a part of a body (organ, tissue, etc.), or a phantom.

Throughout the specification, an ‘ultrasound image’ refers to an image of an object generated or processed based on ultrasound signals transmitted to the object and reflected therefrom.

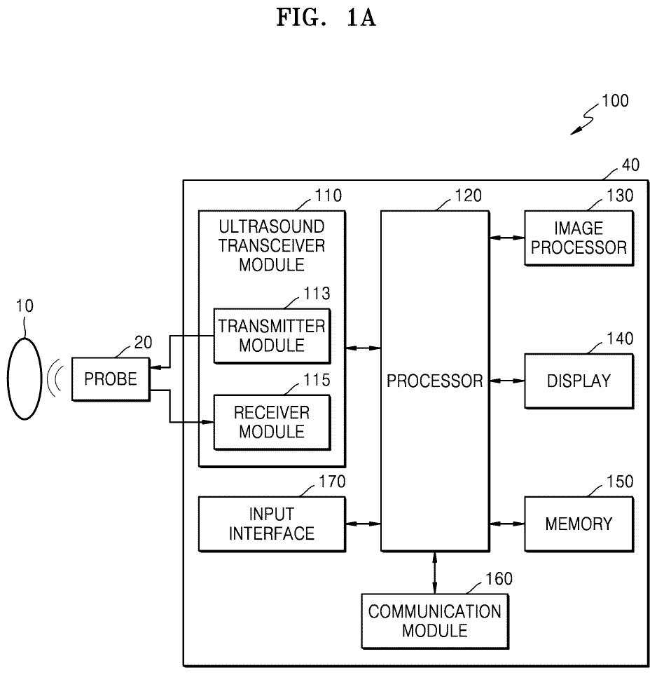

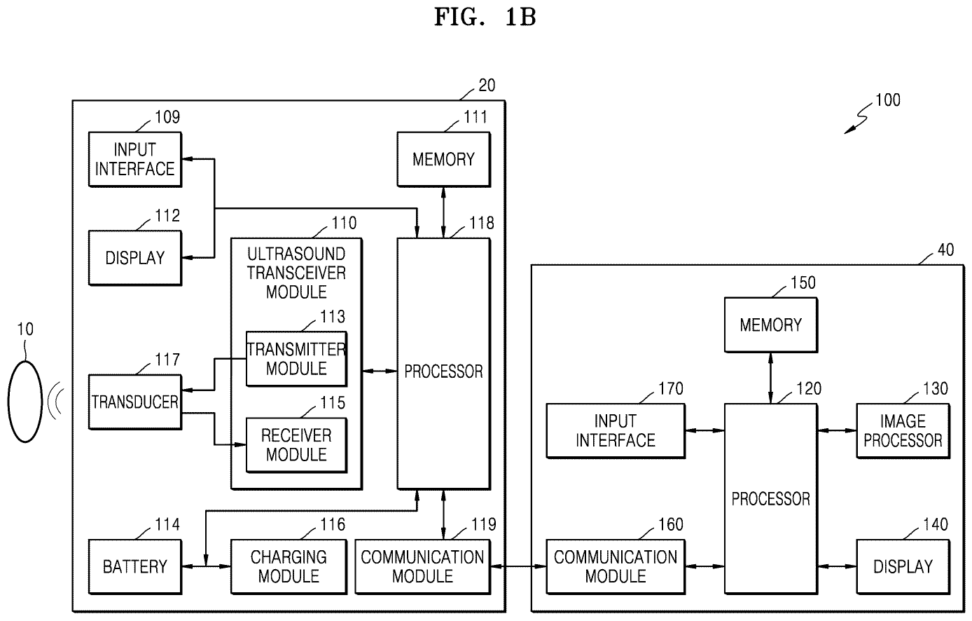

Referring to FIGS. 1A and 1B, an ultrasound imaging system 100 may include a probe 20 and an ultrasound imaging apparatus 40

The ultrasound imaging apparatus 40 may be implemented not only as a cart-type ultrasound imaging apparatus but also as a portable ultrasound imaging apparatus. Examples of the portable ultrasound imaging apparatus may include, but are not limited to, a smartphone, a laptop computer, a personal digital assistant (PDA), a tablet personal computer (PC), etc., each of which includes a probe and an application. The ultrasound imaging apparatus 40 may be formed integrally with the probe 20.

The probe 20 may include a wired probe that is connected to the ultrasound imaging apparatus 40 by wire to communicate with the ultrasound imaging apparatus 40 by wire, a wireless probe that is wirelessly connected to the ultrasound imaging apparatus 40 to communicate wirelessly with the ultrasound imaging apparatus 40, and/or a hybrid probe that is connected to the ultrasound imaging apparatus 40 by wire or wirelessly to communicate with the ultrasound imaging apparatus 40 by wire or wirelessly.

According to various embodiments, the ultrasound imaging apparatus 40 may include an ultrasound transceiver module 110 as shown in FIG. 1A, or the probe 20 may include the ultrasound transceiver module 110 as shown in FIG. 1B. According to various embodiments, the ultrasound imaging apparatus 40 and the probe 20 may both include the ultrasound transceiver module 110.

According to various embodiments, the probe 20 may further include at least one of an image processor 130, a display 140, or an input interface 170, or a combination thereof. In the disclosure, descriptions of the ultrasound transceiver module 110, the image processor 130, the display 140, or the input interface 170 included in the ultrasound imaging apparatus 40 may also apply to the ultrasound transceiver module 110, the image processor 130, the display 140, or the input interface 170 included in the probe 20

FIG. 1A is a block diagram of a configuration of the ultrasound imaging system 100 when the probe 20 is a wired probe or a hybrid probe.

The probe 20 may include a plurality of transducers. The plurality of transducers are arranged in a certain array, forming a transducer array. The transducer array may correspond to a one-dimensional (1D) array or a two-dimensional (2D) array. The plurality of transducers may transmit ultrasound signals to an object 10 in response to transmission signals applied from a transmitter module 113. The plurality of transducers may receive ultrasound (echo) signals reflected from the object 10 to form reception signals. In addition, the probe 20 may be formed integrally with the ultrasound imaging apparatus 40, or may be implemented as a separate part connected to the ultrasound imaging apparatus 40 by wired. In addition, the ultrasound imaging apparatus 40 may be connected to one or a plurality of probes 20 according to its implemented configuration.

When the probe 20 is a wired probe or hybrid probe, the probe 20 may include a cable and a connector that are connectable to a connector of the ultrasound imaging apparatus 40

According to an embodiment, the probe 20 may be implemented as a 2D probe. When the probe 20 is implemented as a 2D probe, the plurality of transducers included in the probe 20 may be arranged in 2D to form a 2D transducer array

For example, the 2D transducer array may include a plurality of sub-arrays, each of the plurality of sub-arrays including a plurality of transducers arranged in a first direction, wherein the plurality of sub-arrays are arranged in a second direction that is different from the first direction.

In addition, according to an embodiment, when the probe 20 is implemented as a 2D probe, the ultrasound transceiver module 110 may include at least one of an analog beamformer or a digital beamformer. In addition, according to an embodiment, the 2D probe may include at least one of an analog beamformer or a digital beamformer, or a combination thereof, according to its implemented configuration.

The processor 120 may control the transmitter module 113 to form transmission signals to be respectively applied to the plurality of transducers based on positions and a focal point of the plurality of transducers.

The processor 120 may control the receiver module 115 to perform analog-to-digital conversion (ADC) on the reception signals received from the probe 20 and generate ultrasound data by summing the digital reception signals based on the positions and the focal point of the plurality of transducers.

When the probe 20 is implemented as a 2D probe, the processor 120 may calculate a time delay value for digital beamforming with respect to each of the plurality of sub-arrays included in the 2D transducer array. In addition, the processor 120 may calculate a time delay value for analog beamforming with respect to each of the plurality of transducers included in any one of the plurality of sub-arrays. The processor 120 may control the analog beamformer and the digital beamformer to form a transmission signal to be applied to each of the plurality of transducers based on time delay values for analog beamforming and digital beamforming. The processor 120 may also control the analog beamformer to sum signals received from the plurality of transducers for each sub-array according to the time delay values for analog beamforming. In addition, the processor 120 may control the ultrasound transceiver module 110 to perform ADC on the resulting sum signal for each sub-array. In addition, the processor 120 may control the digital beamformer to generate ultrasound data by summing the digital output signals according to the time delay values for digital beamforming.

The image processor 130 generates or processes an ultrasound image by using the generated ultrasound data.

The display 140 may display the generated ultrasound image and various pieces of information processed by the ultrasound imaging apparatus 40 or the probe 20. The probe 20 or the ultrasound imaging apparatus 40 may include one or a plurality of displays 140 depending on its implemented configuration. In addition, the display 140 may include a touch panel or a touch screen. The display 140 may also include a flexible display.

The processor 120 may control all operations of the ultrasound imaging apparatus 40 and operations of components of the ultrasound imaging apparatus 40. The processor 120 may execute programs or instructions stored in the memory 150 to perform or control various operations or functions of the ultrasound imaging apparatus 40. The processor 120 may also receive a control signal from the input interface 170 or an external device to control an operation of the ultrasound imaging apparatus 40.

The ultrasound imaging apparatus 40 includes the communication module 160 via which the ultrasound imaging apparatus 40 may be connected to and communicate with an external device (e.g., the probe 20, a server, a medical device, and a portable device such as a smartphone, a tablet PC, a wearable device, etc.)

The communication module 160 may include at least one component that enables communication with an external device. The communication module 160 may include, for example, at least one of a short-range communication module, a wired communication module, or a wireless communication module.

The communication module 160 may receive a control signal and data from an external device. The processor 120 may control an operation of the ultrasound imaging apparatus 40 in response to the control signal received via the communication module 160. In addition, the processor 120 may transmit a control signal to an external device via the communication module 160 to control the external device in response to the transmitted control signal. The external device may operate in response to a control signal received from the ultrasound imaging apparatus 40, or process data received from the ultrasound imaging apparatus 40.

A program or application related to the ultrasound imaging apparatus 40 may be installed on the external device. The program or application installed on the external device may control the ultrasound imaging apparatus 40, or run in response to a control signal or data received from the ultrasound imaging apparatus 40.

The external device may receive or download the program or application related to the ultrasound imaging apparatus 40 from the ultrasound imaging apparatus 40, the probe 20, or a server, and install and execute the program or application thereon. The ultrasound imaging apparatus 40, the probe 20, or the server providing a program or application may include a recording medium storing instructions, commands, installation files, executable files, or related data of the program or application. The external device may also be sold with programs or applications installed.

The memory 150 may store various types of data or programs for driving and controlling the ultrasound imaging apparatus 40, input and/or output ultrasound data, ultrasound images, etc.

The input interface 170 may receive a user input for controlling the ultrasound imaging apparatus 40. For example, the user input may include, but is not limited to, inputs for manipulating buttons, keypads, mice, trackballs, jog switches, or knops, an input for touching a touchpad or a touch screen, a voice input, a motion input, and an input of biometric information (e.g., iris recognition, fingerprint recognition, etc.)

The at least one processor 120 may include a processing circuitry. Instructions in the memory 150 are individually or collectively executed by the at least one processor 120, and thus the ultrasound imaging apparatus 40 may perform an embodiment.

The at least one processor 120 may receive a user input for obtaining an elasticity value of a region of interest (ROI) of an object through the input interface 170.

The at least one processor 120 may repeatedly perform elastography on the ROI through the ultrasound transceiver module 110 based on receiving the user input for obtaining the elasticity value of the ROI of the object.

The at least one processor 120 may obtain an elasticity image of the ROI through elastography. The at least one processor 120 obtains the elasticity image, thereby identifying at least one elasticity measurement ROI on the elasticity image.

The at least one processor 120 may obtain an elasticity value from each of the identified at least one elasticity measurement ROI.

The at least one processor 120 may store each of the obtained elasticity values as elasticity data.

The at least one processor 120 may display information about the number of elasticity values stored as the elasticity data together with the obtained elasticity image through the display 140.

The at least one processor 120 may perform next elastography through the ultrasound transceiver module 110 based on the number of stored elasticity data being less than a target number. The at least one processor 120 may obtain a next elasticity image through next elastography.

The at least one processor 120 may obtain the next elasticity image through next elastography on the ROI based on the number of the elasticity data being less than the target number or a volatility indicator of the elasticity data not satisfying a volatility indicator condition.

The at least one processor 120 may display, on the display 140, information indicating that elasticity data has been sufficiently obtained based on the number of the elasticity data being greater than or equal to the target number and the volatility indicator of the elasticity data satisfying the volatility indicator condition.

The at least one processor 120 may end elastography based on the number of elasticity data greater than or equal to the target number and the volatility indicator of the elasticity data satisfying the volatility indicator condition.

The at least one processor 120 may determine a representative value of the elasticity data as the elasticity value of the ROI based on the number of elasticity data being greater than or equal to the target number and the volatility indicator of the elasticity data satisfying the volatility indicator condition.

The representative value of the elasticity data may be a median of the elasticity data. The volatility indicator may be a ratio between an interquartile range (IQR) and the median of the elasticity data. The volatility indicator condition may be a condition in which the ratio between the IQR and the median of the elasticity data is within a predetermined ratio.

The number of elasticity data exceeds the target number, and thus the at least one processor 120 may select the target number of elasticity values from the elasticity data and store the selected elasticity values as elasticity data. The at least one processor 120 may identify whether the number of stored elasticity data is greater than or equal to the target number and whether the volatility indicator of the stored elasticity data satisfies the volatility indicator condition.

As the elasticity image is obtained, the at least one processor 120 may display, on the display 140, information indicating a ratio of the target number to the number of elasticity data in which elasticity values are accumulated as a progress of measurement of the elasticity value.

As the at least one processor 120 obtains the elasticity image, the at least one processor 120 may display, on the display 140, a volatility indicator of elasticity data in which the elasticity value are accumulated.

The at least one processor 120 may display the elasticity measurement ROI identified on the elasticity image on the display 140.

The at least one processor 120 may receive a user input for deleting an elasticity image through the input interface 170. The at least one processor 120 may exclude an elasticity value which is obtained from the deleted elasticity image from the elasticity data.

FIG. 1B is a control block diagram of a configuration of the ultrasound imaging system 100 when the probe 20 is a wireless probe or a hybrid probe.

According to various embodiments, the ultrasound imaging apparatus 40 shown in FIG. 1B may be replaced with the ultrasound imaging apparatus 40 described with reference to FIG. 1A.

According to various embodiments, the probe 20 shown in FIG. 1A may be replaced with the probe 20 described with reference to FIG. 1B.

The probe 20 may include a display 112, a transmitter module 113, a battery 114, a transducer 117, a charging module 116, a receiver module 115, an input interface 109, a processor 118, and a communication module 119. Although FIG. 1B shows that the probe 20 includes both the transmitter module 113 and the receiver module 115, according to its implemented configuration, the probe 20 may include only some of the components of the transmitter module 113 and the receiver module 115, and the ultrasound imaging apparatus 40 may also include some of the components of the transmitter module 113 and the receiver module 115. In addition, according to an embodiment, the probe 20 may further include the image processor 130.

The transducer 117 may include a plurality of transducers. The plurality of transducers are arranged in a certain array, forming a transducer array. The transducer array may correspond to a 1D array or a 2D array. The plurality of transducers may transmit ultrasound signals to the object 10 in response to transmission signals applied from the transmitter module 113. In addition, the plurality of transducers may receive ultrasound signals reflected from the object 10 to form or generate reception signals.

The charging module 116 may charge the battery 114. The charging module 116 may receive power from an external source. According to an embodiment, the charging module 116 may receive power wirelessly. In addition, according to an embodiment, the charging module 116 may receive power by wired. The charging module 116 may transmit the received power to the battery 114.

The processor 118 may control the transmitter module 113 to generate or form transmission signals to be respectively applied to the plurality of transducers based on positions and a focal point of the plurality of transducers.

The processor 118 may control the receiver module 115 to perform ADC on the reception signals received from the transducer 117 and generate ultrasound data by summing the digital reception signals based on the positions and the focal point of the plurality of transducers. According to an embodiment, when the probe 20 includes the image processor 130, the image processor 130 may generate an ultrasound image based on the generated ultrasound data.

When the probe 20 is implemented as a 2D probe, the processor 118 may calculate a time delay value for digital beamforming with respect to each of the plurality of sub-arrays included in the 2D transducer array. In addition, the processor 118 may calculate a time delay value for analog beamforming with respect to each of the plurality of transducers included in any one of the plurality of sub-arrays. The processor 118 may control the analog beamformer and the digital beamformer to form a transmission signal to be applied to each of the plurality of transducers based on time delay values for analog beamforming and digital beamforming. The processor 120 may also control the analog beamformer to sum signals received from the plurality of transducers for each sub-array according to the time delay values for analog beamforming. In addition, the processor 118 may control the ultrasound transceiver module 110 to perform ADC on the resulting sum signal for each sub-array. In addition, the processor 118 may control the digital beamformer to generate ultrasound data by summing the digital output signals according to the time delay values for digital beamforming.

The processor 118 may control all operations of the probe 20 and operations of components of the probe 20. The processor 118 may execute programs or instructions stored in the memory 111 to perform or control various operations or functions of the probe 20. The processor 118 may also receive a control signal from the input interface 109 of the probe 20 or an external device (e.g., the ultrasound imaging apparatus 40) to control an operation of the probe 20. The processor 118 may also receive a control signal from the input interface 109 or an external device to control an operation of the probe 20. The input interface 109 may receive a user input for controlling the probe 20. For example, the user input may include, but is not limited to, inputs for manipulating buttons, keypads, mice, trackballs, jog switches, or knops, an input for touching a touchpad or a touch screen, a voice input, a motion input, and an input of biometric information (e.g., iris recognition, fingerprint recognition, etc.)

The display 112 may display ultrasound images generated by the probe 20, ultrasound images generated by processing ultrasound data generated by the probe 20, ultrasound images received from the ultrasound imaging apparatus 40, various pieces of information processed by the ultrasound imaging system 100, etc. In addition, the display 112 may further display status information of the probe 20. The status information of the probe 20 may include at least one of device information of the probe 20, battery status information of the probe 20, frequency band information of the probe 20, output information of the probe 20, information about failures of the probe 20, setting information of the probe 20, or temperature information of the probe 20.

The probe 20 may include one or a plurality of displays 112 depending on its implemented configuration. In addition, the display 112 may include a touch panel or a touch screen. The display 112 may also include a flexible display.

The communication module 119 may wirelessly transmit the generated ultrasound data or ultrasound image to the ultrasound imaging apparatus 40 via a wireless network. The communication module 119 may also receive a control signal and data from the ultrasound imaging apparatus 40.

The ultrasound imaging apparatus 40 may receive ultrasound data or an ultrasound image from the probe 20.

In an embodiment, when the probe 20 includes the image processor 130 capable of generating an ultrasound image by using ultrasound data, the probe 20 may transmit ultrasound data or an ultrasound image generated by the image processor 130 to the ultrasound imaging apparatus 40.

In an embodiment, when the probe 20 does not include the image processor 130 capable of generating an ultrasound image by using ultrasound data, the probe 20 may transmit ultrasound data to the ultrasound imaging apparatus 40. Ultrasound data may include ultrasound raw data, and an ultrasound image may mean ultrasound image data.

The ultrasound imaging apparatus 40 may include the processor 120, the image processor 130, the display 140, the memory 150, the communication module 160, and the input interface 170.

The image processor 130 generates or processes an ultrasound image by using ultrasound data received from the probe 20.

The display 140 may display an ultrasound image received from the probe 20, an ultrasound image generated by processing ultrasound data received from the probe 20, various pieces of information processed by the ultrasound imaging system 100, etc. The ultrasound imaging apparatus 40 may include one or a plurality of displays 140 depending on its implemented configuration. In addition, the display 140 may include a touch panel or a touch screen. In addition, the display 140 may include a flexible display.

The processor 120 may control all operations of the ultrasound imaging apparatus 40 and operations of components of the ultrasound imaging apparatus 40. The processor 120 may execute programs or applications stored in the memory 150 to perform or control various operations or functions of the ultrasound imaging apparatus 40. The processor 120 may also receive a control signal from the input interface 170 or an external device to control an operation of the ultrasound imaging apparatus 40.

The ultrasound imaging apparatus 40 includes the communication module 160 via which the ultrasound imaging apparatus 40 may be connected to and communicate with an external device (e.g., the probe 20, a server, a medical device, and a portable device such as a smartphone, a tablet PC, a wearable device, etc.)

The communication module 160 may include at least one component that enables communication with an external device. The communication module 160 may include, for example, at least one of a short-range communication module, a wired communication module, or a wireless communication module.

The communication module 160 of the ultrasound imaging apparatus 40 may communicate with the communication module 119 of the probe 20 by using a network or a short-range wireless communication method. For example, the communication module 160 of the ultrasound imaging apparatus 40 may communicate with the communication module 119 of the probe 20 by using any one of wireless data communication methods including a wireless local area network (WLAN), Wi-Fi, Bluetooth, ZigBee, Wi-Fi Direct (WFD), Infrared Data Association (IrDA), Bluetooth Low Energy (BLE), near field communication (NFC), wireless broadband Internet (WiBro), World Interoperability for Microwave Access (WiMAX), Shared Wireless Access Protocol (SWAP), Wireless Gigabit Alliance (WiGig), radio frequency (RF) communication, 60 gigahertz (GHz) millimeter wave (mmWave) short-range communication, etc.

To this end, the communication module 160 of the ultrasound imaging apparatus 40 and the communication module 119 of the probe 20 may each include at least one of a WLAN communication module, a Wi-Fi communication module, a Bluetooth communication module, a ZigBee communication module, a WFD communication module, an IrDA communication module, a BLE communication module, an NFC communication module, a WiBro) communication module, a WiMAX communication module, a SWAP communication module, a WiGig communication module, an RF communication module, or a 60 GHz mmWave short-range communication module.

In an embodiment, the probe 20 may transmit device information (e.g., identification (ID) information) of the probe 20 to the ultrasound imaging apparatus 40 by using a first communication method (e.g., BLE), and may be paired wirelessly with the ultrasound imaging apparatus 40. In addition, the probe 20 may transmit ultrasound data and/or ultrasound images to the paired ultrasound imaging apparatus 40.

The device information of the probe 20 may include various pieces of information related to a serial number, a model name, a battery status, etc. of the probe 20.

The ultrasound imaging apparatus 40 may receive, from the probe 20, the device information (e.g., ID information) of the probe 20 by using the first communication method (e.g., BLE), and may be paired wirelessly with the probe 20. In addition, the ultrasound imaging apparatus 40 may transmit an activation signal to the paired probe 20 and receive ultrasound data and/or ultrasound images from the probe 20. In this regard, the activation signal may include a signal for controlling an operation of the probe 20.

In addition, the probe 20 may transmit ultrasound data and/or ultrasound images to the ultrasound imaging apparatus 40 paired through the first communication method by using a second communication method (e.g., 60 GHz mmWave or Wi-Fi).

In addition, the ultrasound imaging apparatus 40 may transmit an activation signal to the paired probe 20 and receive ultrasound data and/or ultrasound images from the probe 20 by using the second communication method (e.g., 60 GHz mmWave or Wi-Fi).

According to an embodiment, the first communication method used to pair the probe 20 and the ultrasound imaging apparatus 40 with each other may have a lower frequency band than the second communication method used by the probe 20 to transmit ultrasound data and/or ultrasound images to the ultrasound imaging apparatus 40.

The display 140 of the ultrasound imaging apparatus 40 may display UIs indicating device information of the probe 20. For example, the display 140 may display UIs indicating ID information of the probe 20, a pairing method indicating a method of pairing the ultrasound imaging apparatus 40 with the probe 20, a status of data communication between the probe 20 and the ultrasound imaging apparatus 40, a method of performing data communication with the ultrasound imaging apparatus 40, a battery status of the probe 20, etc.

When the probe 20 includes the display 112, the display 112 of the probe 20 may display a UI indicating device information of the probe 20. For example, the display 112 may display UIs indicating ID information of the probe 20, a pairing method indicating a method of pairing the probe 20 with the ultrasound imaging apparatus 40, a status of data communication between the probe 20 and the ultrasound imaging apparatus 40, a method of performing data communication with the ultrasound imaging apparatus 40, a battery status of the probe 20, etc.

The communication module 160 may receive a control signal and data from an external device. The processor 120 may control an operation of the ultrasound imaging apparatus 40 in response to the control signal received via the communication module 160.

In addition, the processor 120 may transmit a control signal to an external device via the communication module 160 to control the external device in response to the transmitted control signal. The external device may operate in response to a control signal received from the ultrasound imaging apparatus 40, or process data received from the ultrasound imaging apparatus 40.

The external device may receive or download the program or application related to the ultrasound imaging apparatus 40 from the ultrasound imaging apparatus 40, the probe 20, or a server, and install and execute the program or application thereon. The ultrasound imaging apparatus 40, the probe 20, or the server providing a program or application may include a recording medium storing instructions, commands, installation files, executable files, or related data of the program or application. The external device may also be sold with programs or applications installed.

The memory 150 may store various types of data or programs for driving and controlling the ultrasound imaging apparatus 40, input and/or output ultrasound data, ultrasound images, etc.

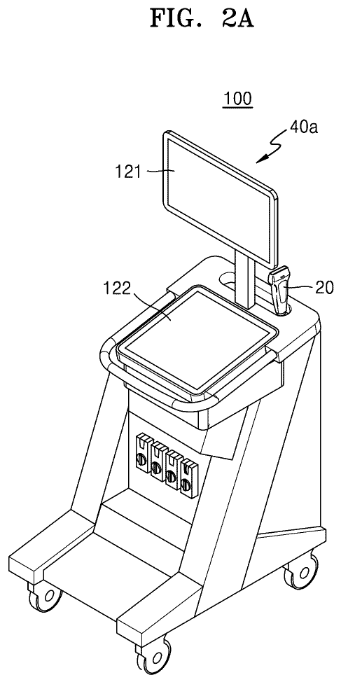

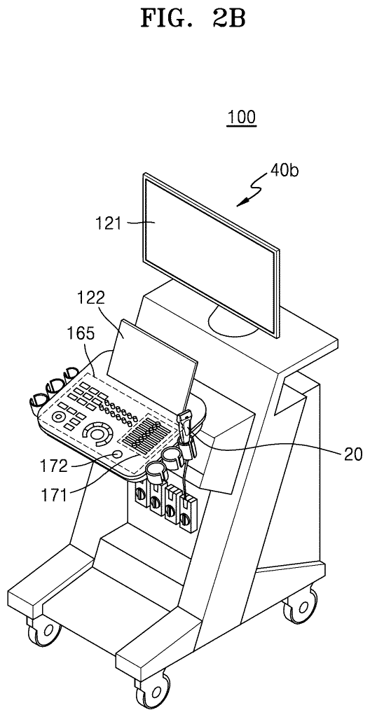

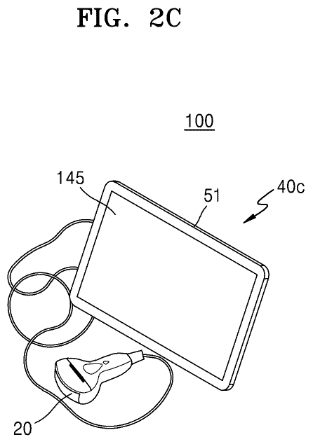

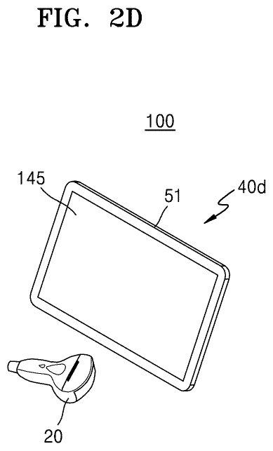

Examples of the ultrasound imaging system 100 according to an embodiment will be described below with reference to FIGS. 2A to 2D.

FIGS. 2A to 2D are diagrams illustrating ultrasound imaging apparatuses 40a to 40d, according to an embodiment.

Referring to FIGS. 2A and 2B, each of the ultrasound imaging apparatuses 40a and 40b may include a main display 121 and a sub-display 122. The main display 121 and the sub-display 122 may correspond to the display 140 of FIGS. 1A and 1B. At least one of the main display 121 or the sub-display 122 may be implemented as a touch screen. At least one of the main display 121 or the sub-display 122 may display an ultrasound image or various pieces of information processed by the ultrasound imaging apparatuses 40a and 40b. In addition, at least one of the main display 121 or the sub-display 122 may be implemented as a touch screen, and may receive data to control the ultrasound imaging apparatuses 40a and 40b from a user by providing a graphic user interface (GUI). For example, the main display 121 may display an ultrasound image, and the sub-display 122 may display a control panel for controlling the displaying of the ultrasound image in a GUI form. The sub-display 122 may receive data to control displaying of the image through the control panel displayed in the form of the GUI. For example, a time grain compensation (TGC) button, a lateral gain compensation (LGC) button, a freeze button, a trackball, a jog switch or a knob may be provided as GUIs on the sub-display 122.

The ultrasound imaging apparatuses 40a and 40b may use the input control data to control displaying of the ultrasound image displayed on the main display 121. In addition, the ultrasound imaging apparatuses 40a and 40b may be connected to the probe 20 by wired or wirelessly to transmit or receive an ultrasound signal to or from an object.

Referring to FIG. 2B, the ultrasound imaging apparatus 40b may further include a control panel 165 in addition to the main display 121 and the sub-display 122. The control panel 165 may include a button, a trackball, a jog switch, a knob, etc., and may receive data to control the ultrasound imaging apparatus 40b from the user. For example, the control panel 165 may include a TGC button 171, a freeze button 172, etc. The TGC button 171 is a button for setting a TGC value for each depth of the ultrasound image. In addition, when an input to the freeze button 172 is detected while scanning the ultrasound image, the ultrasound imaging apparatus 40b may maintain displaying a frame image at the time, capture the frame image at the time, or store the frame image at the time.

In the meantime, the button, the trackball, the jog switch, the knob, etc., included in the control panel 165 may be provided as GUIs on the main display 121 or the sub-display 122. The ultrasound imaging apparatuses 40a and 40b may also be connected to the probe 20 to transmit or receive an ultrasound signal to or from the object.

The ultrasound imaging apparatuses 40a and 40b may include various types of input/output interfaces such as a speaker, a light emitting diode (LED), a vibration device, etc. For example, the ultrasound imaging apparatuses 40a and 40b may output various pieces of information through the input/output interface in such a form as graphics, sound or vibration. The ultrasound imaging apparatuses 40a and 40b may also output various types of notifications or data through the input/output interface.

Referring to FIGS. 2C and 2D, the ultrasound imaging apparatuses 40c and 40d may be implemented in a portable type. Examples of the portable-type ultrasound imaging apparatuses 40c and 40d may include a smart phone, a laptop computer, a PDA or a tablet PC, which includes a probe and an application, but are not limited thereto.

The ultrasound imaging apparatus 40c may include a main body 411. Referring to FIG. 2C, the probe 20 may be connected to a side of the main body 41 by wired. To this end, the main body 41 may include a connection terminal to or from which a cable connected to the probe 20 may be attached or detached. The probe 20 may include the cable that includes a connection terminal to be connectable to the main body 41.

Referring to FIG. 2D, the probe 20 may be wirelessly connected to the ultrasound imaging apparatus 40d. The main body 41 may include an input/output interface (e.g., a touch screen). The input/output interface may display an ultrasound image, various information processed by the ultrasound imaging apparatus 40d or a GUI.

The ultrasound imaging apparatus 40d and the probe 20 may establish communication or may be paired by a short-range wireless communication. For example, the ultrasound imaging apparatus 40d and the probe 20 may use Bluetooth, BLE, Wi-Fi or Wi-Fi direct to communicate with each other.

The ultrasound imaging apparatuses 40c and 40d may execute a program or application related to the probe 20 to control the probe 20 and output information relating to the probe 20. The ultrasound imaging apparatuses 40c and 40d may perform an operation related to the probe 20 while communicating with a certain server. The probe 20 may be registered in the ultrasound imaging apparatuses 40c and 40d or in the certain server. The ultrasound imaging apparatuses 40c and 40d may communicate with the registered probe 20 and perform an operation related to the probe 20.

In addition, the ultrasound imaging apparatuses 40c and 40d may include various types of input/output interfaces such as a speaker, an LED, a vibration device, etc. For example, the ultrasound imaging apparatuses 40c and 40d may output various information through the input/output interface in such a form as graphics, sound or vibration. The ultrasound imaging apparatuses 40c and 40d may output various types of notifications or data through the input/output interface.

In an embodiment, the ultrasound imaging apparatus 40a, 40b, 40c or 40d may use an artificial intelligence (AI) model to process the ultrasound image or obtain additional information from the ultrasound image. In an embodiment, the ultrasound imaging apparatus 40a, 40b, 40c or 40d may use the AI model to generate an ultrasound image or perform such a process as correction, image quality enhancement, encoding or decoding on the ultrasound image. In addition, in an embodiment, the ultrasound imaging apparatus 40a, 40b, 40c or 40d may use the AI model to perform a process such as defining a baseline, obtaining anatomical information, obtaining lesion information, extracting a surface, defining boundary, measuring a length, measuring an area, measuring a volume or generating an annotation from the ultrasound image.

The AI model may be provided on the ultrasound imaging apparatus 40a, 40b, 40c or 40d or on a server.

The AI model may be implemented by using various artificial neural networks or deep neural networks. The AI model may also be trained or generated by using various machine learning algorithms or deep learning algorithms. The AI model may be implemented, for example, by using a model, such as a convolutional neural network (CNN), a recurrent neural network (RNN), a generative adversarial network (GAN), long short-term memory (LSTM), etc.

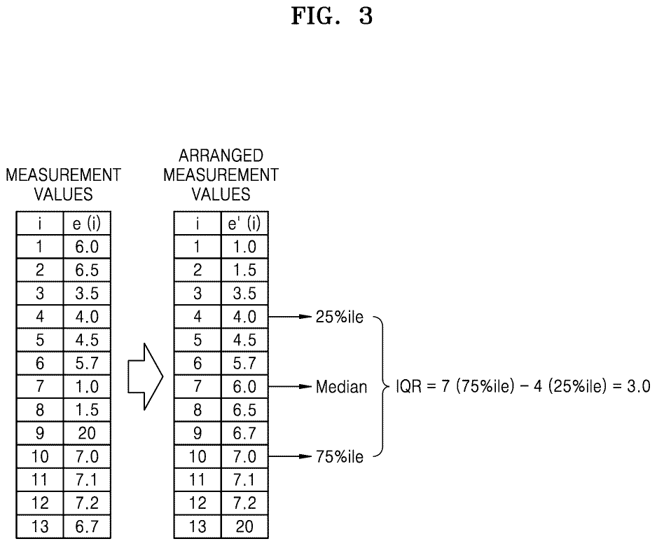

FIG. 3 illustrates a method, performed by the ultrasound imaging apparatus 40, of determining an elasticity value of a ROI, according to an embodiment.

Referring to FIG. 3, the ultrasound imaging apparatus 40 may accumulate elasticity data by repeatedly obtaining an elasticity image of the ROI, and determine a representative value of the elasticity data as the elasticity value of the ROI.

Measurement values of the same region need theoretically be the same. However, even though the elasticity value is repeatedly obtained with respect to the same region, there may be a large difference between elasticity values due to the movement of an object or the movement of a user. Therefore, it is necessary to repeatedly measure more than a reference number of times with respect to the same region in order to increase the accuracy of measurement of the elasticity value. For example, 10 or more repeated measurements may be required for the accuracy of measurement of the elasticity value.

In addition, the elasticity data may include outliers that significantly deviate from normal elasticity values due to the movement of the object. Accordingly, the representative value of the elasticity data may be a median that is less affected by outliers. However, the disclosure is not limited thereto, and, for example, the representative value of the elasticity data may be a trimmed mean.

The ultrasound imaging apparatus 40 may exclude the outliers from the elasticity values in the elasticity data and calculate, as a volatility indicator, a degree to which the elasticity data excluding the outliers varies with respect to the representative value. The greater the volatility of the elasticity data, the greater the volatility indicator may be. The ultrasound imaging apparatus 40 may increase the reliability of the representative value by repeating elastography until the volatility indicator of the elasticity data is less than or equal to a reference value.

The volatility indicator may be calculated, for example, as a ratio of the median to an intermediate range of the elasticity data. The intermediate range of the elasticity data may be a value obtained by subtracting a value corresponding to the bottom 25% from a value corresponding to the top 75% of the elasticity data, but is not limited thereto. The ratio (e.g., IQR/Med) of the median to the intermediate range of the elasticity data may indicate a degree to which the elasticity data, excluding the outliers, is spread with respect to the median.

In addition, the volatility indicator may include, for example, a trimmed variance or a trimmed standard deviation.

The user may be required to repeat measurement of the elasticity value until the volatility indicator is less than or equal to the reference value. For example, the user may be required to repeat measurement of the elasticity value until the ratio (IQR/Med) of the median of the elasticity data to an interquartile range (IQR) indicating the intermediate 50% of the elasticity data is less than or equal to a reference ratio (e.g., 0.3).

Referring back to FIG. 3, the ultrasound imaging apparatus 40 may repeatedly obtain the elasticity image, and obtain 13 elasticity values from the obtained elasticity images as the elasticity data. When the elasticity data are arranged in order of magnitude, a value corresponding to the bottom 25% is 4.0, and a value corresponding to the top 75% is 7.0, and thus the IQR of the elasticity data is 3.0 which is a value by subtracting 4 from 7. The median of the elasticity data is 6.0, and 0.2( 3/6) which is the ratio of the median to the intermediate range of the elasticity data is lower than the reference ratio of 0.3, and thus the ultrasound imaging apparatus 40 may determine 6.0 which is the median of the elasticity data as the elasticity value of the ROI.

While the elasticity image is repeatedly obtained, the object may be required to minimize its movement. For example, while an elasticity image of the liver is repeatedly obtained to measure stiffness of the liver, a patient may be required to stop breathing with space between the ribs open by raising his/her arm above his/her head so as to minimize the movement of the liver.

While the elasticity image is repeatedly obtained, because it is difficult to know whether elasticity values greater than or equal to the target number are accumulated, or whether the volatility indicator of the elasticity data is less than or equal to the reference value, the user repeats elastography with his or her experience value.

When the number of elasticity data is less than the target number or the volatility indicator exceeds the reference value after finishing elastography, the user may have to repeat elastography again. In addition, even though the number of elasticity data is greater than or equal to the target number and the volatility indicator is less than the reference value, the user may not recognize whether the condition has been satisfied and continuously perform elastography. Because the patient may be required to stop breathing during elastography, additional elastography or elastography time more than necessary may be a significant burden on the patient.

According to an embodiment, the ultrasound imaging apparatus 40 may display information indicating that the elasticity data has been sufficiently obtained when the number of elasticity values accumulated as the elasticity data is greater than or equal to the target number and the volatility indicator of the elasticity data is less than or equal to the reference value while repeatedly obtaining the elasticity image.

According to an embodiment, the ultrasound imaging apparatus 40 may determine the progress of measurement of the elasticity value as the elasticity values are accumulated, and display the determined progress of measurement of the elasticity value.

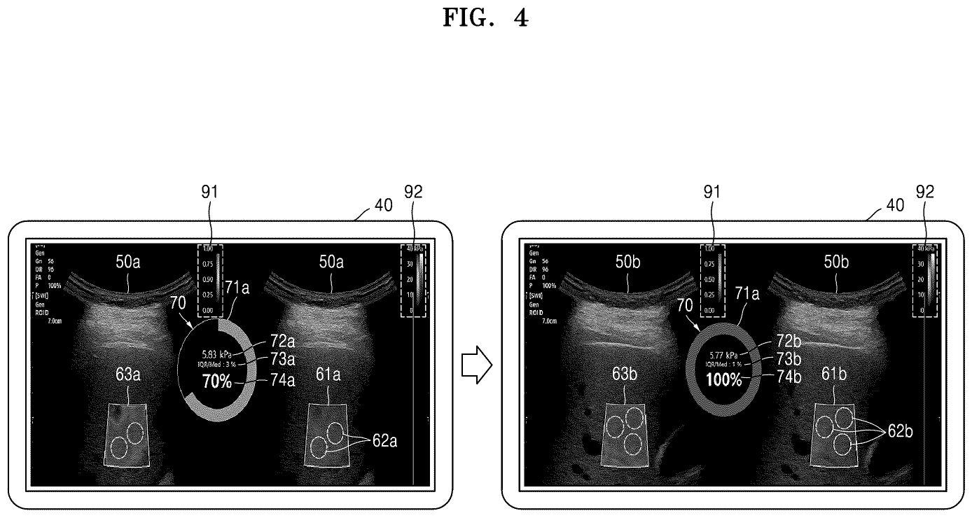

FIG. 4 illustrates a method, performed by the ultrasound imaging apparatus 40, of providing information about a progress 74a of measurement of an elasticity value, according to an embodiment.

Referring to FIG. 4, the ultrasound imaging apparatus 40 may display information indicating that elasticity data has been sufficiently obtained when the number of elasticity values accumulated as the elasticity data is greater than or equal to a target number and a volatility indicator of the elasticity data is less than or equal to a reference value while repeatedly obtaining an elasticity image.

Referring to the left drawing of FIG. 4, the ultrasound imaging apparatus 40 may display an elasticity image 61a, a reliability image 63a, and information 70 about elasticity data on a B mode image 50a.

For example, the ultrasound imaging apparatus 40 may obtain a B-mode image (not shown) of an object through a B-mode scan after a patient takes a posture. The ultrasound imaging apparatus 40 may receive a user input for setting an elasticity imaging region on the B mode image (not shown). The ultrasound imaging apparatus 40 may irradiate a strong ultrasound impulse to tissue of an object corresponding to the elasticity imaging region.

The ultrasound imaging apparatus 40 may repeatedly perform elastography. In one elastography, the ultrasound imaging apparatus 40 may generate an ultrasound shear wave in the tissue by irradiating the strong ultrasound impulse to the tissue corresponding to the elasticity imaging region.

After irradiating the strong ultrasound impulse, the ultrasound imaging apparatus 40 may obtain ultrasound data from the elasticity imaging region by irradiating an ultrasound wave for high-speed frame capturing to the elasticity imaging region, detect changes in the positions of tissues caused by the ultrasound shear wave from the obtained ultrasound data, and calculate the speed at which the ultrasound shear wave moves based on the detected changes in the positions of tissues. The ultrasound imaging apparatus 40 may determine elasticity values of the elasticity imaging region based on the propagation speed of the ultrasound shear wave and generate the elasticity image 61a. In addition, the ultrasound imaging apparatus 40 may generate the B mode image 50a of the object by irradiating ultrasound waves for B mode scanning to the object.

The elasticity image (61a) can represent elasticity values at each point using colors. For example, the elasticity values in the elasticity image (61a) may range from 0 to 40 kPa, and the elasticity image (61a) may represent the elasticity values ranging from 0 to 40 kPa with colors in the order of blue, light blue, yellow, and red. Accordingly, stiff tissues may be represented in red, indicating high energy, while soft tissues may be represented in blue, indicating low energy. The ultrasound imaging apparatus (40) may display an elasticity color bar (92) together with the elasticity image (61a). The elasticity color bar (92) may indicate the colors shown in the elasticity image (61a) and the corresponding elasticity values.

In addition, the ultrasound imaging apparatus 40 may obtain the reliability image 63a indicating the reliability of elasticity values in the elasticity imaging region. For example, the ultrasound imaging apparatus 40 may determine the magnitude of the ultrasound shear wave, the signal quality of the ultrasound shear wave, or a degree of noise of the ultrasound shear wave based on the obtained ultrasound data. In addition, the ultrasound imaging apparatus 40 may generate the reliability image 63a indicating the reliability of the elasticity values represented by the elasticity image 61a based on the magnitude of the ultrasound shear wave, the signal quality of the ultrasound shear wave, or the degree of noise of the ultrasound shear wave. The ultrasound imaging apparatus 40 may determine that the larger the magnitude of the induced shear wave, the higher the signal quality of the shear wave, and the smaller the noise level of the shear wave, the higher the reliability of the elasticity value. The reliability image 63a may express the reliability in a color. For example, the lower the reliability of the elasticity value, the reliability may be in a red color, and the higher the reliability of the elasticity value, the reliability may be in a green color. Additionally, the ultrasound imaging apparatus (40) may display the reliability image (63a) and a reliability color bar (91). The reliability color bar (91) may indicate the colors shown in the reliability image (63a) and the corresponding reliability values.

As the ultrasound imaging apparatus 40 obtains the elasticity image 61a, the ultrasound imaging apparatus 40 may obtain at least one elasticity value from the obtained elasticity image 61a as elasticity data. For example, the ultrasound imaging apparatus 40 may determine at least one elasticity measurement ROI 62a in which the reliability of the elasticity value is greater than or equal to the reference value based on the reliability image 63a and obtain an elasticity value from each of the at least one elasticity measurement ROI 62a.

The ultrasound imaging apparatus 40 may accumulate the obtained at least one elasticity value as the elasticity data. In addition, the ultrasound imaging apparatus 40 may determine a representative value 72a of the elasticity data and a volatility indicator 73a of the elasticity data based on the accumulated elasticity data. For example, the ultrasound imaging apparatus 40 may determine the representative value 72a of the elasticity data to be 5.83 kPa which is a median of the elasticity data. In addition, the ultrasound imaging apparatus 40 may determine the determined volatility indicator 73a of the elasticity data to be 3% which is an IQR/Med value.

The ultrasound imaging apparatus 40 may display the progress 74a of measurement of the elasticity value. For example, the ultrasound imaging apparatus 40 may determine the progress 74a of measurement of the elasticity value as the number of accumulated elasticity values compared to the target number of elasticity values. For example, when the target number of elasticity values is 10 and the accumulated number of elasticity values is 7, the ultrasound imaging apparatus 40 may determine the progress 74a of measurement of the elasticity value to be 70%.

The ultrasound imaging apparatus 40 may display the representative value 72a of the elasticity data, the volatility indicator 73a of the elasticity data, and the progress 74a of measurement of the elasticity value.

In addition, the ultrasound imaging apparatus 40 may determine whether elastography for obtaining the elasticity value is further required. Based on the elasticity values included in the elasticity data being not obtained more than the target number, the ultrasound imaging apparatus 40 may determine that elastography for obtaining the elasticity value is further required.

As the ultrasound imaging apparatus 40 determines that elastography is further required, the ultrasound imaging apparatus 40 may again irradiate a strong ultrasound impulse on the tissue to generate an ultrasound shear wave in the tissue, and obtain new ultrasound data of an object in which the ultrasound shear wave has occurred. The ultrasound imaging apparatus 40 may generate an elasticity image 61b and a reliability image 63b based on the obtained ultrasound data. In addition, the ultrasound imaging apparatus 40 may generate a B mode image 50b of the object by irradiating an ultrasound wave for B mode scanning to the object.

As the ultrasound imaging apparatus 40 obtains the elasticity image 61b, the ultrasound imaging apparatus 40 may determine at least one elasticity measurement ROI 62b in the obtained elasticity image 61b, obtain at least one elasticity value from the at least one elasticity measurement ROI 62b, and accumulate the obtained at least one elasticity value as elasticity data. In addition, the ultrasound imaging apparatus 40 may determine a representative value 72b of the elasticity data and a volatility 73b of the elasticity data based on the accumulated elasticity data.

In addition, based on the IQR/Med value indicating the volatility of elasticity data being 1%, which satisfies a volatility indicator condition of 30% or less, and the number of accumulated elasticity values being 10, which is greater than or equal to the target number of elasticity values of 10, the ultrasound imaging apparatus 40 may determine a progress 74b of measurement of the elasticity value to be 100%.

The ultrasound imaging apparatus 40 may display the representative value 72b of the elasticity data, the volatility 73b of the elasticity data, and the progress 74b of measurement of the elasticity value.

In addition, the ultrasound imaging apparatus 40 may determine that elasticity data has been sufficiently obtained and elastography for obtaining an elasticity value is no longer required, based on the number of elasticity data being greater than or equal to the target number and the volatility of elasticity data being less than or equal to the reference value.

According to an embodiment, the ultrasound imaging apparatus 40 may stop elastography based on determining that elastography for obtaining an elasticity value is no longer required.

According to an embodiment, based on determining that elastography for obtaining an elasticity value is no longer required, the ultrasound imaging apparatus 40 may display information indicating that elasticity data has been sufficiently obtained, but may continue elastography to further obtain an elasticity value until a user input for stopping elastography is received.

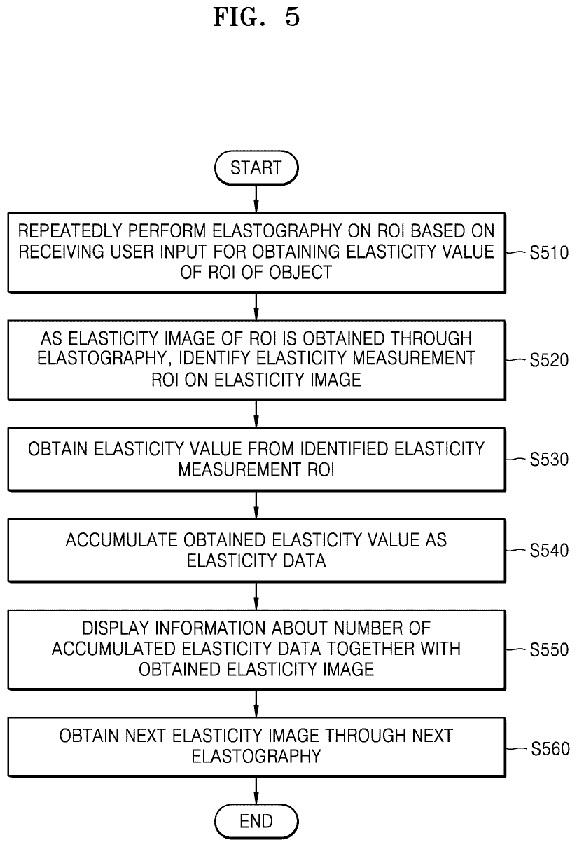

FIG. 5 is a flowchart of a method, performed by the ultrasound imaging apparatus 40, of measuring an elasticity value, according to an embodiment.

In operation S510, the ultrasound imaging apparatus 40 may repeatedly perform elastography on a ROI based on receiving a user input for obtaining an elasticity value of the ROI of an object.

For example, as the ultrasound imaging apparatus 40 receives a user input, the ultrasound imaging apparatus 40 may repeatedly obtain an elasticity image by performing elastography at a predetermined period.

According to an embodiment, the user input for obtaining the elasticity value of the ROI of the object may include a user input for selecting an elastography menu and a user input for selecting an elasticity imaging region.

According to an embodiment, the user input for obtaining the elasticity value of the ROI of the object may include a user input for selecting the elastography menu, a user input for selecting the elasticity imaging region, and a user input for starting elastography.

In operation S520, as the elasticity image of the ROI is obtained through elastography, the ultrasound imaging apparatus 40 may identify an elasticity measurement ROI on the elasticity image.

The elasticity measurement ROI may be a region in which an elasticity value is to be obtained in the elasticity image.

Whenever the ultrasound imaging apparatus 40 obtains an elasticity image, the ultrasound imaging apparatus 40 may identify the elasticity measurement ROI on the elasticity image. For example, the ultrasound imaging apparatus 40 may determine at least one elasticity measurement ROI based on reliability of elasticity values in the elasticity image and uniformity of elasticity values.

The ultrasound imaging apparatus 40 may determine the at least one elasticity measurement ROI in the elasticity image, and display the position of the determined at least one elasticity measurement ROI on the elasticity image.

The ultrasound imaging apparatus 40 may obtain a plurality of elasticity values from one elasticity image, or may not obtain the elasticity value.

In operation S530, the ultrasound imaging apparatus 40 may obtain an elasticity value from the identified elasticity measurement ROI.

The ultrasound imaging apparatus 40 may obtain an elasticity value from each of the at least one elasticity measurement ROI. For example, the ultrasound imaging apparatus 40 may obtain an average value of elasticity values in one elasticity measurement ROI as the elasticity value of the elasticity measurement ROI.

In operation S540, the ultrasound imaging apparatus 40 may accumulate the obtained elasticity value as elasticity data.

Whenever the ultrasound imaging apparatus 40 obtains an elasticity value, the ultrasound imaging apparatus 40 may accumulate the obtained elasticity value as the elasticity data.

In operation S550, the ultrasound imaging apparatus 40 may display information about the number of accumulated elasticity data together with the obtained elasticity image.

As the obtained elasticity value is accumulated as the elasticity data, the ultrasound imaging apparatus 40 may display the information about the number of accumulated elasticity data together with the obtained elasticity image. By providing the number of accumulated elasticity data, a user may determine a progress of obtaining the elasticity value.

According to an embodiment, the ultrasound imaging apparatus 40 may display the obtained elasticity image and display the identified elasticity measurement ROI on the displayed elasticity image.

In operation S560, the ultrasound imaging apparatus 40 may obtain a next elasticity image through next elastography.

The ultrasound imaging apparatus 40 may obtain a new elasticity value from the next elasticity image and accumulate the obtained new elasticity value as elasticity data again. For example, the ultrasound imaging apparatus 40 may perform elastography again based on the number of accumulated elasticity data being less than a target number.

The ultrasound imaging apparatus 40 may display information indicating that elasticity data has been sufficiently obtained based on the number of elasticity data being greater than or equal to the target number and a volatility indicator of the elasticity data satisfying a volatility indicator condition.

The target number may be selected by the user. For example, the ultrasound imaging apparatus 40 may provide a user interface capable of selecting the target number, and may receive a user input for setting the target number through the user interface.

The volatility indicator may mean a degree to which elasticity data changes with respect to a representative value. The greater the volatility of the elasticity data, the greater the volatility indicator may be. The volatility indicator condition may be that the volatility indicator is less than or equal to a reference value. The ultrasound imaging apparatus 40 may increase the reliability of the representative value of the elasticity data in consideration of the volatility of the elasticity data as well as the number of elasticity data.

According to an embodiment, the representative value of elasticity data may be a median of elasticity data. The volatility indicator may be a ratio (IQR/Med) between the IQR value and the median of the elasticity data. The volatility indicator condition may be a condition in which the IQR/Med of the elasticity data is less than or equal to the reference value.

The ultrasound imaging apparatus 40 may obtain the next elasticity image through next elastography on the ROI based on the number of elasticity data being less than the target number or the volatility indicator of the elasticity data not satisfying the volatility indicator condition. Even though the number of elasticity data is greater than or equal to the target number, when the volatility indicator of the elasticity data does not satisfy the volatility indicator condition, the ultrasound imaging apparatus 40 may continue elastography.

According to an embodiment, the ultrasound imaging apparatus 40 may stop elastography based on the number of elasticity data being greater than or equal to the target number and the volatility indicator of the elasticity data satisfying the volatility indicator condition. As elastography is stopped, the ultrasound imaging apparatus 40 may determine a representative value of the elasticity data as an elasticity value of the ROI.

According to an embodiment, the ultrasound imaging apparatus 40 may proceed with next elastography without stopping elastography until there is a user input for stopping elastography based on the number of elasticity data being greater than or equal to the target number and the volatility indicator of the elasticity data satisfying the volatility indicator condition.

According to an embodiment, the ultrasound imaging apparatus 40 may provide a user interface for setting whether to stop elastography when the number of elasticity data being greater than or equal to the target number and the volatility indicator of the elasticity data satisfying the volatility indicator condition. The ultrasound imaging apparatus 40 may receive a user input for setting whether to stop elastography when elasticity data satisfying the volatility indicator condition is obtained more than the target number through the user interface. The ultrasound imaging apparatus 40 may determine whether to stop elastography according to a setting of the user.

According to an embodiment, as the number of elasticity data exceeds the target number, the ultrasound imaging apparatus 40 may select the target number of elasticity values from the elasticity data, and update only the selected elasticity values as elasticity data. The ultrasound imaging apparatus 40 may select elasticity values in which a difference between representative values among the elasticity data is less than or equal to a reference difference value. The ultrasound imaging apparatus 40 may identify again whether the volatility indicator of the elasticity data satisfies the volatility indicator condition based on the updated elasticity data.

As the ultrasound imaging apparatus 40 repeatedly obtains the elasticity image, the ultrasound imaging apparatus 40 may display the progress of measurement of the elasticity value. For example, the ultrasound imaging apparatus 40 may display information indicating a ratio of the target number to the number of accumulated elasticity data as the progress of measurement of the elasticity value.

Even though the number of elasticity data is greater than or equal to the target number, when the volatility indicator of the elasticity data does not satisfy the volatility indicator condition, the ultrasound imaging apparatus 40 may not determine the progress of measurement of the elasticity value to be 100%. For example, even though the number of elasticity data is greater than or equal to the target number, when the volatility indicator of the elasticity data does not satisfy the volatility indicator condition, the ultrasound imaging apparatus 40 may increase the target number and calculate the progress based on the increased target number.

Whenever the ultrasound imaging apparatus 40 obtains an elasticity image, the ultrasound imaging apparatus 40 may display a volatility indicator of the updated elasticity data.

As the ultrasound imaging apparatus 40 repeatedly performs elastography on the ROI, the ultrasound imaging apparatus 40 may display a list of elasticity images for which elasticity values have been obtained.

The ultrasound imaging apparatus 40 may exclude an elasticity value which is obtained from a deleted elasticity image from the elasticity data based on receiving a user input for deleting the displayed elasticity image.

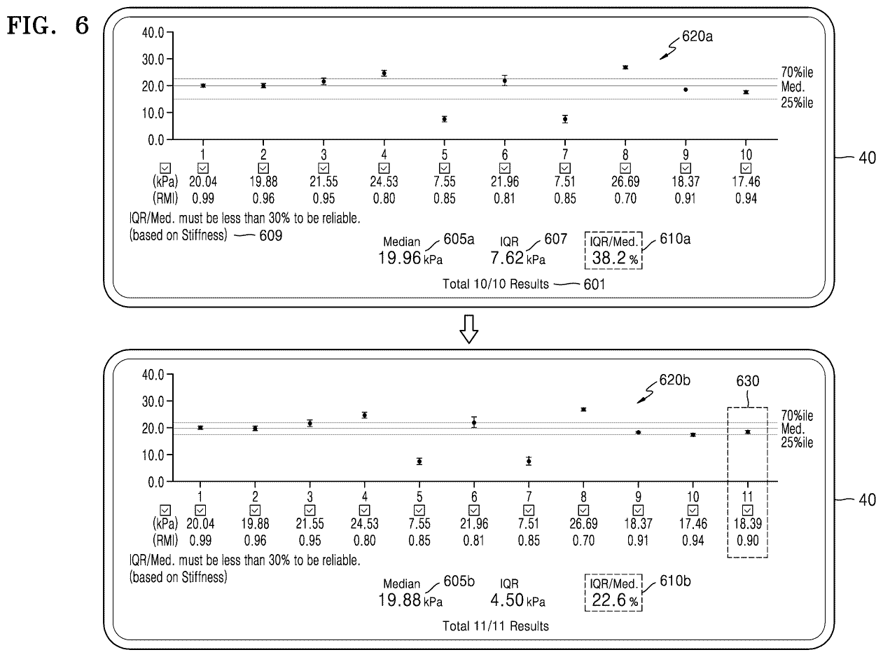

FIG. 6 illustrates a method, performed by the ultrasound imaging apparatus 40, of additionally obtaining elasticity data based on volatility indicators 610a and 610b of elasticity data, according to an embodiment.

Referring to FIG. 6, when the volatility of the elasticity data does not satisfy a volatility indicator condition, the ultrasound imaging apparatus 40 may continue elastography for obtaining an elasticity value.

Referring to the upper drawing of FIG. 6, the ultrasound imaging apparatus 40 may store a plurality of elasticity values obtained from a plurality of elasticity images as elasticity data through repeated capturing of a ROI.

The ultrasound imaging apparatus 40 may display the elasticity data as an elasticity value graph 620a. For example, when obtaining an elasticity image, the ultrasound imaging apparatus 40 may display the elasticity value graph 620a together with the elasticity image. In addition, for example, the ultrasound imaging apparatus 40 may provide a user interface for displaying the elasticity data. Based on receiving a user input for selecting a user interface, the ultrasound imaging apparatus 40 may display the elasticity value graph 620a.

The elasticity value graph 620a may represent a median and an IQR, and may represent a degree to which elasticity values are separated from the median.

In addition, the ultrasound imaging apparatus 40 may display the volatility indicator 610a of the elasticity data. The ultrasound imaging apparatus 40 may display a number 601 of the obtained elasticity values. The ultrasound imaging apparatus 40 may display information 609 about the volatility indicator condition of the elasticity data. The volatility indicator condition of the elasticity data may be, for example, a condition in which IQR/Med is less than 30%. Although not shown in FIG. 6, the ultrasound imaging apparatus 40 may display the target number of elasticity values. The target number may be, for example, 10.

A representative value of the elasticity data may be 19.96 kPa, which is a median 605a of the elasticity data. An IQR value 607 may be 7.62 kPa, and the volatility indicator 610a of the elasticity data may be 38.2% which is an IQR/Med value.

Even though the number of elasticity values obtained as the elasticity data satisfies the target number, the ultrasound imaging apparatus 40 may continue elastography for obtaining an elasticity value when the volatility indicator 610a of the elasticity data exceeds a volatility indicator reference value.

Referring to FIG. 6, although the number of elasticity values is 10 which satisfies the target number, because the volatility indicator 610a of the elasticity data is 38.2%, which does not satisfy the volatility indicator condition of less than 30%, the ultrasound imaging apparatus 40 may obtain a next elasticity image. The ultrasound imaging apparatus 40 may obtain an eleventh elasticity value 630 from the obtained next elasticity image. The ultrasound imaging apparatus 40 accumulate the eleventh elasticity value 630 as elasticity data, and recalculate a representative value 605b of the elasticity data and the volatility indicator 610b of the elasticity data. As the volatility indicator 610b of the elasticity data falls to 22.6% which is less than the volatility indicator condition of 30%, the ultrasound imaging apparatus 40 may display information indicating that the elasticity values more than the target number have been obtained and that a representative value of the elasticity data satisfies the volatility indicator condition. The ultrasound imaging apparatus 40 may determine the representative value of the elasticity data as an elasticity value of a ROI, and may obtain no further elasticity image.

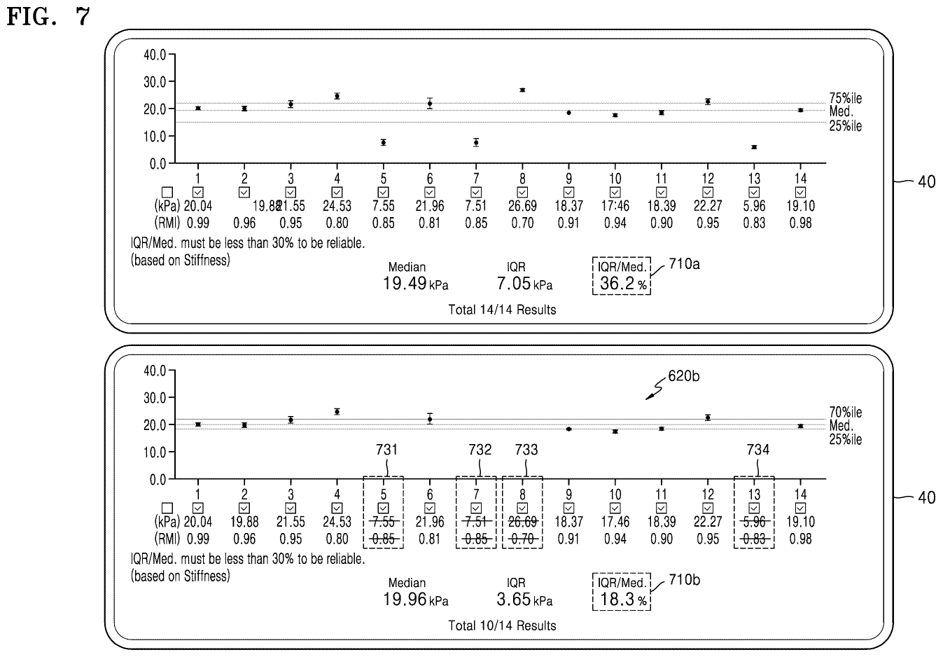

FIG. 7 illustrates a method, performed by the ultrasound imaging apparatus 40, of determining a representative value of elasticity data by excluding outliers 731, 732, 733, and 734, according to an embodiment.

Referring to FIG. 7, the ultrasound imaging apparatus 40 may exclude the outliers 731, 732, 733, and 734 from the elasticity data based on a volatility of the elasticity data not satisfying a volatility indicator condition even though the number of obtained elasticity values has reached a threshold number.

For example, referring to the upper drawing of FIG. 7, an IQR/Med 710a of the elasticity data may be 36.2% which does not satisfy the volatility indicator condition of 30% or less even though the number of elasticity data exceeds a target number and has reached a threshold number of 14. The threshold number may be predetermined and stored to be greater than or equal to the target number, and may be changed according to a user input.

The ultrasound imaging apparatus 40 may identify outliers among the elasticity data. For example, the ultrasound imaging apparatus 40 may identify, as outliers, a predetermined number of elasticity values in order of distance from the representative value. In addition, for example, the ultrasound imaging apparatus 40 may identify, as outliers, values less than Q1 (values at the bottom 25% point)−1.5*IQR or greater than Q3 (values at the top 25% point)+1.5*IQR among the elasticity data with respect to an IQR.

Referring to the lower drawing of FIG. 7, the ultrasound imaging apparatus 40 may recalculate a volatility indicator of the elasticity data excluding the outliers 731, 732, 733, and 734. As an IQR/Med 710b of the elasticity data is 18.3% which satisfies the volatility indicator condition of 30% or less, the ultrasound imaging apparatus 40 may no longer perform elastography and determine a median of the elasticity data as an elasticity value of an ROI.

An elastography time may be unnecessarily increased due to outliers caused by a movement of an object. The ultrasound imaging apparatus 40 may remove the outliers 731, 732, 733, and 734 from the elasticity data and not perform additional elastography, thereby reducing the burden on the object.

According to an embodiment, based on the volatility indicator of the elasticity data not satisfying the volatility indicator condition even though the outliers 731, 732, 733, and 734 have been removed, the ultrasound imaging apparatus 40 may display a notification to guide additional capturing.

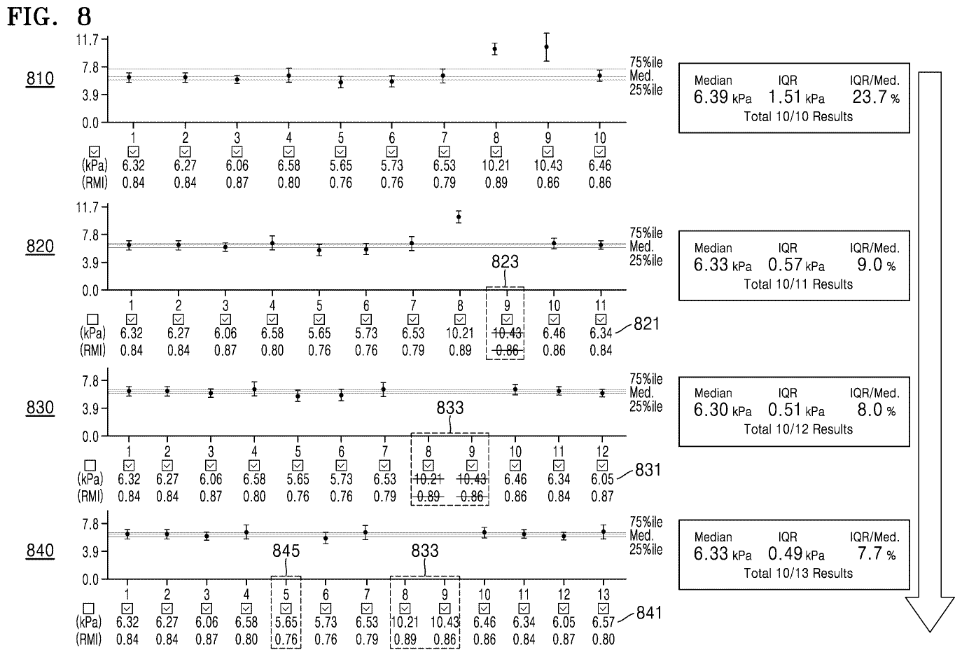

FIG. 8 illustrates a method, performed by the ultrasound imaging apparatus 40, of selecting a target number of elasticity values from elasticity data, according to an embodiment.

Referring to FIG. 8, the ultrasound imaging apparatus 40 may select the target number of elasticity values from the elasticity data based on a distance from a representative value, and calculate a volatility indicator of each of the representative value and the elasticity value based on the selected elasticity values.

Referring to a first drawing 810 of FIG. 8, the ultrasound imaging apparatus 40 may accumulate 10 elasticity values, which are the target number, as the elasticity data from a plurality of elasticity images, and calculate IQR/Med of the elasticity data as 23.7%.

Referring to a second drawing 820 of FIG. 8, as elastography is continued, the ultrasound imaging apparatus 40 may obtain a first elasticity image following the plurality of elasticity images, and obtain an eleventh elasticity value 821 from the first elasticity image. The ultrasound imaging apparatus 40 may select the 10 elasticity values, which are the target number, from among the 11 elasticity values, and exclude a ninth elasticity value 823 which is the farthest from a median from the elasticity data. As the ninth elasticity value 823 is excluded, the ultrasound imaging apparatus 40 may calculate IQR/Med of the remaining 10 elasticity values as 9.0%, thereby reducing the volatility of the elasticity data.

Referring to a third drawing 830 of FIG. 8, the ultrasound imaging apparatus 40 may obtain a second elasticity image following the first elasticity image and obtain a twelfth elasticity value 831 from the second elasticity image. The ultrasound imaging apparatus 40 may select the 10 elasticity values, which are the target number, from among the 12 elasticity values, and exclude eighth and ninth elasticity values 833 that are the farthest from the median from the elasticity data. As the eighth and ninth elasticity values 833 are excluded, the ultrasound imaging apparatus 40 may calculate the IQR/Med of the remaining 10 elasticity values as 8.0%, thereby further reducing the volatility of the elasticity data.

Referring to a fourth drawing 840 of FIG. 8, the ultrasound imaging apparatus 40 may obtain a third elasticity image following the second elasticity image and obtain a thirteenth elasticity value 841 from the third elasticity image. The ultrasound imaging apparatus 40 may select the 10 elasticity values, which are the target number, from among the 13 elasticity values, and exclude a fifth elasticity value 845 and the eighth and ninth elasticity values 833, which are the farthest from the median. As the fifth elasticity value 845 and the eighth and ninth elasticity values 833 are excluded, the ultrasound imaging apparatus 40 may calculate the IQR/Med of the remaining 10 elasticity values as 7.7%, thereby further reducing the volatility of the elasticity data.

As the elasticity data is updated, the ultrasound imaging apparatus 40 may display a representative value of the elasticity value and a volatility indicator of the elasticity value.

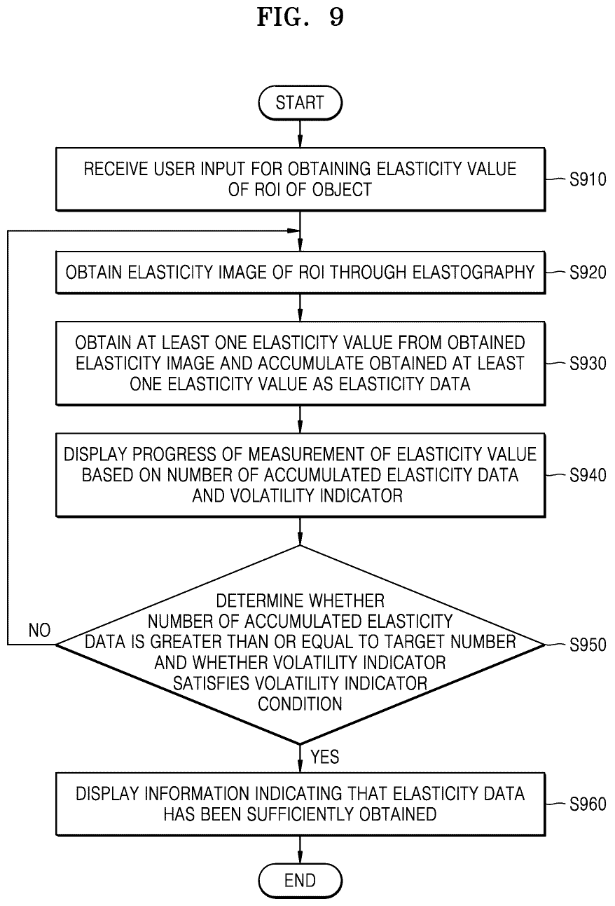

FIG. 9 is a flowchart of a method, performed by the ultrasound imaging apparatus 40, of displaying a progress of measurement of an elasticity value, according to an embodiment.

In operation S910, the ultrasound imaging apparatus 40 may receive a user input for obtaining an elasticity value of a ROI of an object. Operation S910 may be described with reference to operation S510 of FIG. 5.

In operation S920, the ultrasound imaging apparatus 40 may obtain an elasticity image of the ROI through elastography.

The ultrasound imaging apparatus 40 may generate an ultrasound shear wave in a tissue by irradiating a strong ultrasound pulse to the tissue corresponding to an elasticity imaging region. The ultrasound imaging apparatus 40 may determine elasticity values of the elasticity imaging region based on a propagation speed of the ultrasound shear wave and generate the elasticity image.

In operation S930, the ultrasound imaging apparatus 40 may obtain at least one elasticity value from the obtained elasticity image and accumulate the obtained at least one elasticity value as elasticity data. Operation S930 may be described with reference to operations S520 and S540 of FIG. 5.

In operation S940, the ultrasound imaging apparatus 40 may display the progress of measurement of the elasticity value based on the number of accumulated elasticity data and a volatility indicator.

The ultrasound imaging apparatus 40 may display information indicating a ratio of a target number to the number of accumulated elasticity data as the progress of measurement of the elasticity value. For example, when the target number is 10 and the number of accumulated elasticity data is 5, the ultrasound imaging apparatus 40 may display 50% as the progress of measurement of the elasticity value.

In operation S950, the ultrasound imaging apparatus 40 may determine whether the number of accumulated elasticity data is greater than or equal to the target number and whether the volatility indicator satisfies a volatility indicator condition.

For example, the volatility indicator may be IQR/Med, and the volatility indicator condition may be a condition in which an IQR/Med value is 0.3 or less.

Based on determining in operation S950 that the number of accumulated elasticity data is greater than or equal to the target number and that the volatility indicator satisfies the volatility indicator condition, in operation S960, the ultrasound imaging apparatus 40 may display information indicating that the elasticity data has been sufficiently obtained.

The information indicating that the elasticity data has been sufficiently obtained may include information indicating that the progress of measurement of the elasticity value is 100%.

According to an embodiment, the ultrasound imaging apparatus 40 may display a representative value of the elasticity data as an elasticity value of a ROI without further performing elastography based on determining that the elasticity data has been sufficiently obtained.

Based on the number of accumulated elasticity data being less than the target number or the volatility indicator not satisfying the volatility indicator condition in operation S950, the ultrasound imaging apparatus 40 may return to operation S920 to obtain a next elasticity image of the ROI through next elastography.

In addition, even though the number of elasticity data is greater than or equal to the target number, when the volatility indicator of the elasticity data does not satisfy the volatility indicator condition, the ultrasound imaging apparatus 40 may not display the progress of measurement of the elasticity value as 100%. For example, even though the number of elasticity data is greater than or equal to the target number, when the volatility indicator of the elasticity data does not satisfy the volatility indicator condition, the ultrasound imaging apparatus 40 may maintain the progress calculated from a previous elasticity image. In addition, for example, even though the number of elasticity data is greater than or equal to the target number, when the volatility indicator of the elasticity data does not satisfy the volatility indicator condition, the ultrasound imaging apparatus 40 may increase the target number and display a ratio of the increased target number to the number of accumulated elasticity data as the progress.

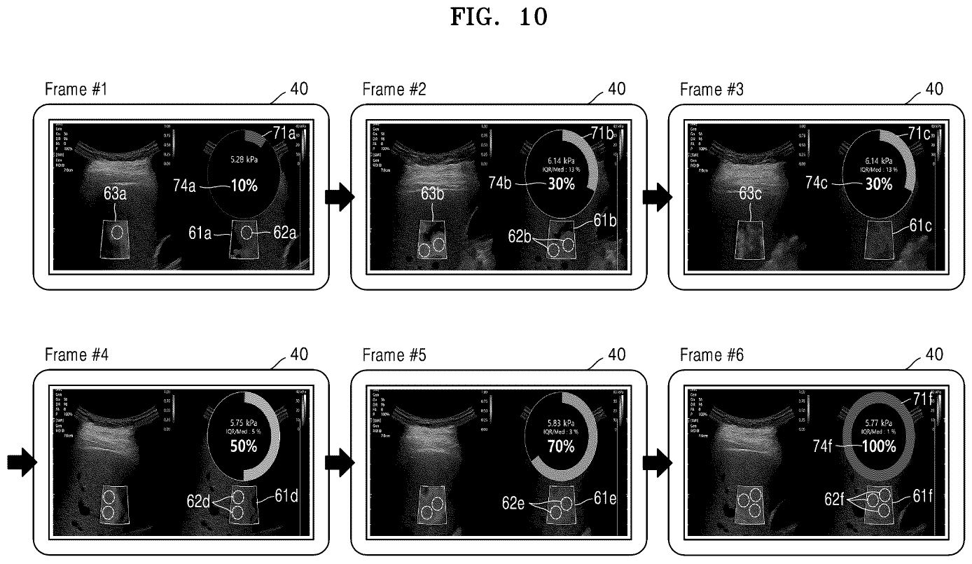

FIG. 10 illustrates a method, performed by the ultrasound imaging apparatus 40, of updating a progress of measurement of an elasticity value, according to an embodiment.

Referring to FIG. 10, whenever an elasticity image is obtained, the ultrasound imaging apparatus 40 may update the progress of measurement of the elasticity value and display the updated progress together with the obtained elasticity image.

Referring to a first drawing of FIG. 10, as the first elasticity image 61a is obtained, the ultrasound imaging apparatus 40 may determine the elasticity measurement ROI 62a to obtain the elasticity value in the first elasticity image 61a based on the reliability 63a of elasticity values in the first elasticity image 61a. As one elasticity value is obtained as elasticity data from one elasticity measurement ROI 62a, the ultrasound imaging apparatus 40 may determine the progress of measurement of the elasticity value.

For example, when a target number is 10, the ultrasound imaging apparatus 40 may determine, as the progress, 10% which is a ratio of the target number (10) to the number (1) of elasticity data. In addition, the ultrasound imaging apparatus 40 may indicate the determined progress as a numerical value 74a and a graph 71a.

Referring to a second drawing of FIG. 10, as the second elasticity image 61b is obtained, the ultrasound imaging apparatus 40 may determine two elasticity measurement ROIs 62b based on the reliability 63b of the second elasticity image 61b, obtain an elasticity value from each of the two elasticity measurement ROIs 62b, and accumulate each elasticity value as elasticity data. The ultrasound imaging apparatus 40 may determine, as the progress, 30% which is a ratio of the target number (10) to the number (3) of elasticity data, and display the determined progress as a numerical value 74b and a graph 71b.

Referring to a third drawing of FIG. 10, as a third elasticity image 61c is obtained, the ultrasound imaging apparatus 40 may display the third elasticity image 61c, but may not determine an elasticity measurement ROI in the third elasticity image 61c based on regions in the third elasticity image 61c having no elasticity region satisfying a reference condition. The reference condition may be, for example, a condition in which reliability values of an elasticity region having a predetermined size are greater than or equal to a reference value.

As the elasticity measurement ROI is not determined, the ultrasound imaging apparatus 40 may maintain the progress of measurement of the elasticity value at 30%, which is a progress of the previous elasticity image 61b.

Referring to a fourth drawing of FIG. 10, as a fourth elasticity image 61d is obtained, the ultrasound imaging apparatus 40 may determine two elasticity measurement ROIs 62d, and determine the progress of measurement of the elasticity value to be 50%. Referring to a fifth drawing of FIG. 10, as a fifth elasticity image 61e is obtained, the ultrasound imaging apparatus 40 may determine two elasticity measurement ROIs 62e, and determine the progress of measurement of the elasticity value to be 70%.

Referring to the last drawing of FIG. 10, as a sixth elasticity image 61f is obtained, the ultrasound imaging apparatus 40 may determine three elasticity measurement ROIs 62f, obtain three elasticity values from three elasticity measurement ROIs 62f, and accumulate the obtained three elasticity values as elasticity data. As the obtained three elasticity values are accumulated as the elasticity data, the number of elasticity data may reach the target number of 10.

When the number of elasticity data is greater than or equal to the target number, the ultrasound imaging apparatus 40 may identify whether a volatility indicator of the elasticity data satisfies a volatility indicator condition. Based on the volatility indicator of the elasticity data being 1% which is less than or equal to 30%, the ultrasound imaging apparatus 40 may determine, as the progress, 100% which is a ratio of the target number (10) to the number (10) of elasticity data, and display the determined progress as a numerical value 74f and a graph 71f.

Although not shown in FIG. 10, according to an embodiment, the ultrasound imaging apparatus 40 may determine the progress of measurement of the elasticity value to be less than 100% based on the volatility indicator of the elasticity data not satisfying the volatility indicator condition even though the number of elasticity data is greater than or equal to the target number.

For example, even though the number of elasticity data is greater than or equal to the target number, based on the volatility indicator of the elasticity data exceeding 30%, the ultrasound imaging apparatus 40 may maintain 70% which is the progress in the previous elasticity image 61e without updating the progress of measurement of the elasticity value.

In addition, for example, even though the number of elasticity data is greater than or equal to the target number, the ultrasound imaging apparatus 40 may increase the target number by a predetermined number based on the volatility indicator of the elasticity data exceeding 30%. For example, when the predetermined number is two, the ultrasound imaging apparatus 40 may increase the target number from 10 to 12. As the target number is changed, the ultrasound imaging apparatus 40 may determine, as the progress, 83%, which is a ratio of the target number (12) to the number (10) of elasticity data, and display the determined progress as a numerical value and a graph.

In addition, the ultrasound imaging apparatus 40 may determine that additional elastography is necessary for obtaining an elasticity value, and may obtain a next elasticity image.

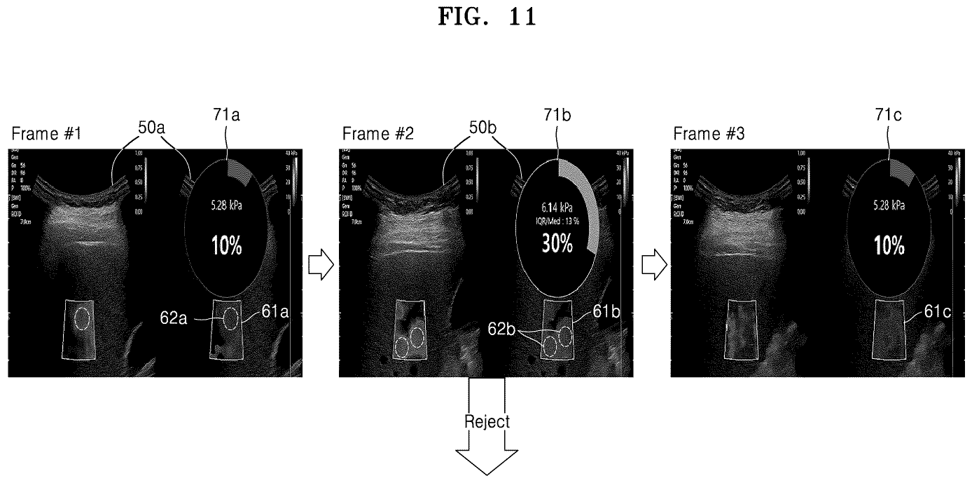

FIG. 11 illustrates a method, performed by the ultrasound imaging apparatus 40, of determining a progress of measurement of an elasticity value based on a user input excluding elasticity images, according to an embodiment.

Referring to FIG. 11, the ultrasound imaging apparatus 40 may receive a user input for excluding one 61b of the plurality of obtained elasticity images 61a, 61b, and 61c.

Whenever an elasticity image is obtained, the ultrasound imaging apparatus 40 may display the obtained elasticity image. The ultrasound imaging apparatus 40 may receive a user input excluding one 61b of the plurality of displayed elasticity images 61a, 61b, and 61c.

The ultrasound imaging apparatus 40 may obtain the third elasticity image 61c after the first elasticity image 61a and the second elasticity image 61b. When the third elasticity image 61c is obtained, the ultrasound imaging apparatus 40 may receive a user input excluding the second elasticity image 61b.

The ultrasound imaging apparatus 40 may exclude the two elasticity values of the second elasticity image 61b from the elasticity data among the three elasticity values corresponding to the elasticity measurement ROIs 62a and 62b obtained from the first elasticity image 61a and the second elasticity image 61b. In addition, the ultrasound imaging apparatus 40 may not obtain an elasticity value from the third elasticity image 61c based on regions in the third elasticity image 61c having no elasticity region satisfying a reference condition.

Accordingly, the ultrasound imaging apparatus 40 may determine only the elasticity value 62a of the elasticity measurement ROI 62a obtained from the first elasticity image 61a as elasticity data, and calculate, as the progress, 10% which is a ratio of the target number of 10 to the number of elasticity data of 1.

The ultrasound imaging apparatus 40 may display the calculated progress of the measurement of the elasticity value on the third elasticity image 61c.

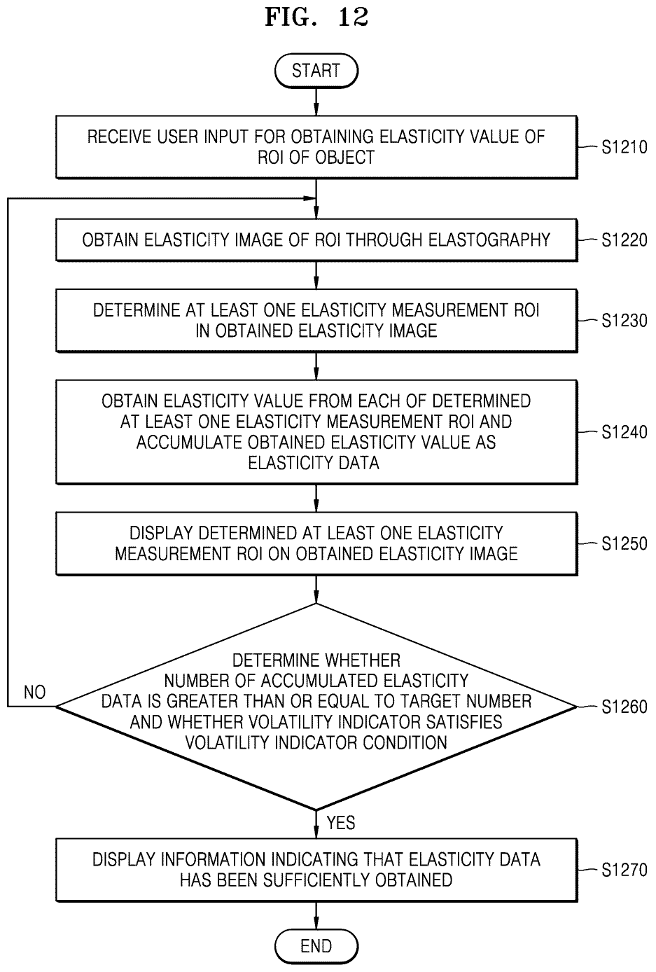

FIG. 12 is a flowchart of a method, performed by the ultrasound imaging apparatus 40, of displaying an elasticity measurement ROI on an elasticity image when the ultrasound imaging apparatus 40 obtains the elasticity image, according to an embodiment.

In operation S1210, the ultrasound imaging apparatus 40 may receive a user input for obtaining an elasticity value of a ROI of an object. In operation S1220, the ultrasound imaging apparatus 40 may obtain an elasticity image of the ROI through elastography. Operations S1210 and S1220 may be described with reference to operations S910 and S920 of FIG. 9.

In operation S1230, the ultrasound imaging apparatus 40 may determine at least one elasticity measurement ROI in the obtained elasticity image.

The ultrasound imaging apparatus 40 may identify a region in which an elasticity value is not structurally measurable among regions in the elasticity image. The region in which an elasticity value is not structurally measurable may be a blood vessel, a cyst, or a shadowing region, but is not limited thereto.

The ultrasound imaging apparatus 40 may exclude the region in which the elasticity value is not structurally measurable among regions in the elasticity image, and then determine a region in which reliability of elasticity values is greater than or equal to a reference value as an elasticity value measurement candidate region.

The ultrasound imaging apparatus 40 may determine at least one elasticity measurement ROI based on the reliability of elasticity values, the uniformity of elasticity values, and the size and shape of the elasticity measurement ROI in the elasticity value measurement candidate region.

The shape of the elasticity measurement ROI may be circular or square, but is not limited thereto. For example, the ultrasound imaging apparatus 40 may determine the shape of the elasticity measurement ROI with respect to each elasticity image based on the reliability of the elasticity values and the uniformity of the elasticity values. In addition, for example, the ultrasound imaging apparatus 40 may receive a user input for inputting a boundary of the elasticity measurement ROI.

In addition, the size of the elasticity measurement ROI may be a predetermined size and adjusted according to a user input. The size of the elasticity measurement ROI may be set to be smaller than the size of the elasticity image.

In operation S1240, the ultrasound imaging apparatus 40 may obtain an elasticity value from each of the determined at least one elasticity measurement ROI, and accumulate the obtained elasticity value as elasticity data.

In operation S1250, the ultrasound imaging apparatus 40 may display the determined at least one elasticity measurement ROI on the obtained elasticity image.

The ultrasound imaging apparatus 40 may display the at least one elasticity measurement ROI on a reliability image. Accordingly, a user may check reliability values of the region in which the elasticity value is obtained.

In operation S1260, the ultrasound imaging apparatus 40 may determine whether the number of accumulated elasticity data is greater than or equal to the target number and whether a volatility indicator satisfies a volatility indicator condition.

Based on determining in operation S1260 that the number of accumulated elasticity data is greater than or equal to the target number and that the volatility indicator satisfies the volatility indicator condition, in operation S1270, the ultrasound imaging apparatus 40 may display information indicating that the elasticity data has been sufficiently obtained.

Based on the number of accumulated elasticity data being less than the target number or the volatility indicator not satisfying the volatility indicator condition in operation S1260, the ultrasound imaging apparatus 40 may return to operation S1220 to perform next elastography and obtain a next elasticity image of the ROI.

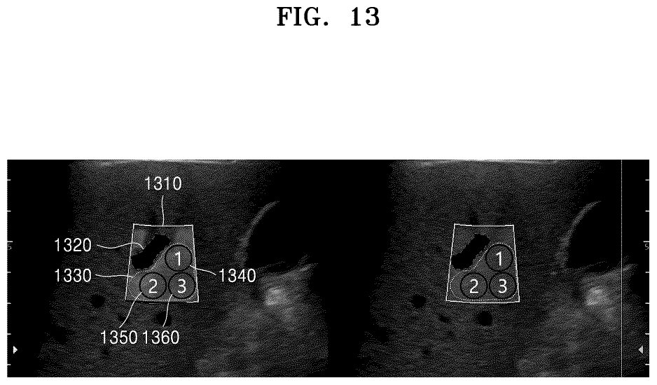

FIG. 13 illustrates a method, performed by the ultrasound imaging apparatus 40, of determining an elasticity measurement ROI, according to an embodiment.

Referring to FIG. 13, the ultrasound imaging apparatus 40 may determine at least one effective elasticity measurement ROI 1340, 1350, and 1360 to obtain an elasticity value in an obtained reliability image 1310.

First, the ultrasound imaging apparatus 40 may determine an elasticity value measurement candidate region 1330 in the obtained reliability image 1310. The ultrasound imaging apparatus 40 may identify a region 1320 in which an elasticity value is not structurally measurable among regions in the reliability image 1310. Referring to FIG. 13, the ultrasound imaging apparatus 40 may identify, as the region 1320 in which the elasticity value is not structurally measurable, a region (e.g., a region close to zero) in which the elasticity value is less than or equal to a reference value in elasticity imaging regions. The region 1320 in which the elasticity value is not structurally measurable may be, for example, a blood vessel region, a cyst region, or a shadowing region. The ultrasound imaging apparatus 40 may determine, as the elasticity value measurement candidate region 1330, a region in which the reliability of elasticity values is greater than or equal to the reference value among regions in the reliability image 1310 excluding the region 1320 in which the elasticity value is not structurally measurable.

The ultrasound imaging apparatus 40 may determine the at least one effective elasticity measurement ROI 1340, 1350, and 1360 in the elasticity value measurement candidate region 1330.

For example, the ultrasound imaging apparatus 40 may determine the at least one effective elasticity measurement ROI 1340, 1350, and 1360 in the elasticity value measurement candidate area 1330 in consideration of the size and shape of the elasticity measurement ROI.

The ultrasound imaging apparatus 40 may determine, as the elasticity measurement ROI, an effective elasticity measurement ROI in which an average value of reliability of the elasticity values is greater than or equal to a reliability reference value and uniformity of the elasticity values is greater than or equal to a uniformity reference value among the at least one effective elasticity measurement ROI 1340, 1350, and 1360.

When only the first effective elasticity measurement ROI 1340 and the second effective elasticity measurement ROI 1350 of the three effective elasticity measurement ROIs 1340, 1350, and 1360 satisfy a reliability condition and a uniformity condition, the ultrasound imaging apparatus 40 may determine the first effective elasticity measurement ROI 1340 and the second effective elasticity measurement ROI 1350 as the elasticity measurement ROIs.

According to an embodiment, the number (e.g., three) of elasticity values that are obtainable from one elasticity image may be set in the ultrasound imaging apparatus 40 to prevent deflection of measurement of the elasticity value. The ultrasound imaging apparatus 40 may determine at least one elasticity measurement ROI based on the set number of elasticity values.

As two elasticity measurement ROIs are determined, the ultrasound imaging apparatus 40 may display the two elasticity measurement ROIs on the elasticity image. In addition, the ultrasound imaging apparatus 40 may obtain an elasticity value from each of the two elasticity measurement ROIs and accumulate each of the obtained elasticity values as elasticity data. The ultrasound imaging apparatus 40 may obtain an average of the elasticity values in the elasticity measurement ROI as an elasticity value of the elasticity measurement ROI.

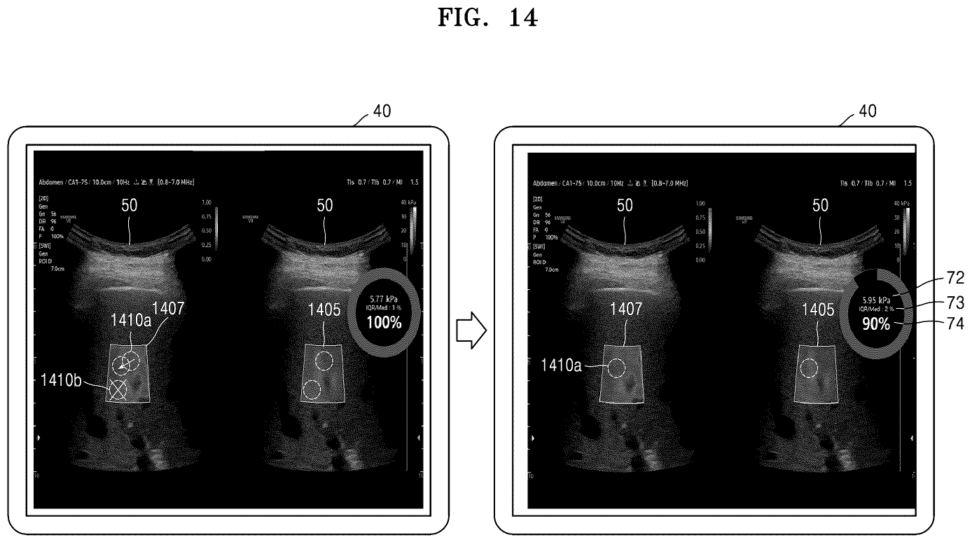

FIG. 14 illustrates a method, performed by the ultrasound imaging apparatus 40, of changing an elasticity measurement ROI based on a user input, according to an embodiment.

Referring to a left drawing of FIG. 14, the ultrasound imaging apparatus 40 may receive a user input for changing a position of the elasticity measurement ROI or removing the elasticity measurement ROI.

As an elasticity image 1405 and a reliability image 1407 are obtained, the ultrasound imaging apparatus 40 may determine first and second elasticity measurement ROIs 1410a and 1410b in the reliability image 1407, and obtain elasticity values corresponding to the first and second elasticity measurement ROIs 1410a and 1410b.

As each elasticity value is obtained, the ultrasound imaging apparatus 40 may determine a progress of measurement of the elasticity value to be 100%.

As a user input for selecting the first elasticity measurement ROI 1410a or the second elasticity measurement ROI 1410b is received, the ultrasound imaging apparatus 40 may display at least one of an average value of elasticity values in the first elasticity measurement ROI 1410a or the second elasticity measurement ROI 1410b, a standard deviation of elasticity values, a minimum value of elasticity values, a maximum value of elasticity values, an average value of reliability values, or a size of a diameter of the first elasticity measurement ROI 1410a or the second elasticity measurement ROI 1410b.

In addition, the ultrasound imaging apparatus 40 may receive a user input for moving the first elasticity measurement ROI 1410a on the reliability image 1407 or the elasticity image 1405. In addition, the ultrasound imaging apparatus 40 may receive a user input for deleting the second elasticity measurement ROI 1410b on the reliability image 1407 or the elasticity image 1405.

Referring to a right drawing of FIG. 14, the ultrasound imaging apparatus 40 may reduce a progress 74 from 100% to 90% based on receiving the user input for deleting the second elasticity measurement ROI 1410b.

In addition, based on deleting the second elasticity measurement ROI 1410b and receiving the user input for moving the first elasticity measurement ROI 1410a, the ultrasound imaging apparatus 40 may recalculate and display a median 72 of the elasticity data and a volatility indicator 73.

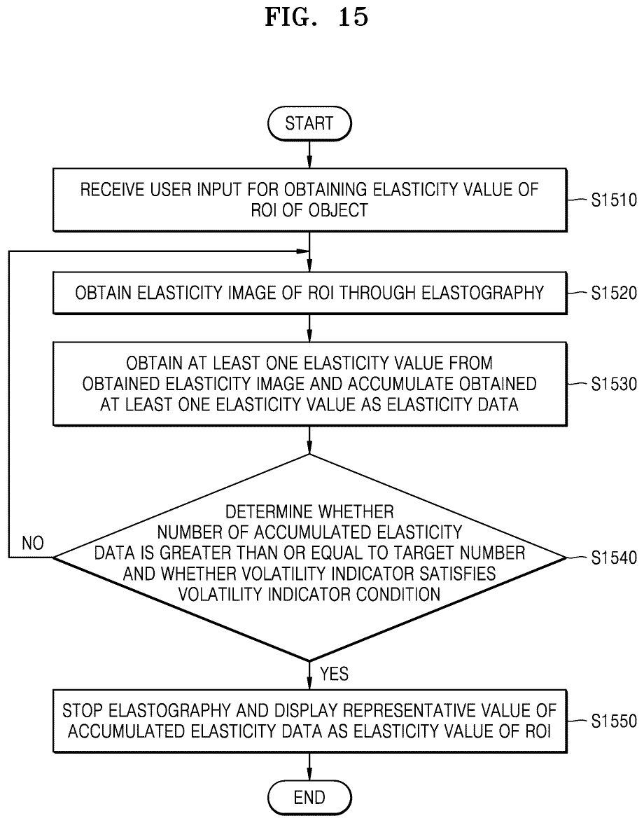

FIG. 15 is a flowchart of a method, performed by the ultrasound imaging apparatus 40, of stopping elastography based on a progress of measurement of an elasticity value, according to an embodiment.

In operation S1510, the ultrasound imaging apparatus 40 may receive a user input for obtaining an elasticity value of a ROI of an object. In operation S1520, the ultrasound imaging apparatus 40 may obtain an elasticity image of the ROI through elastography. In operation S1530, the ultrasound imaging apparatus 40 may obtain at least one elasticity value from the obtained elasticity image and accumulate the obtained at least one elasticity value as elasticity data.

Operations S1510 to S1530 may be described with reference to operations S510 to S540 of FIG. 5.

In operation S1540, the ultrasound imaging apparatus 40 may determine whether the number of accumulated elasticity data is greater than or equal to a target number and whether a volatility indicator satisfies a volatility indicator condition.

Based on determining in operation S1540 that the number of accumulated elasticity data is greater than or equal to the target number and that the volatility indicator satisfies the volatility indicator condition, in operation S1550, the ultrasound imaging apparatus 40 may stop elastography and display a representative value of the accumulated elasticity data as the elasticity value of the ROI. The ultrasound imaging apparatus 40 may display information indicating that elasticity data has been sufficiently obtained.

Based on the number of accumulated elasticity data being less than the target number or the volatility indicator not satisfying the volatility indicator condition in operation S1540, the ultrasound imaging apparatus 40 may return to operation S1520 to obtain a next elasticity image of the ROI through next elastography.

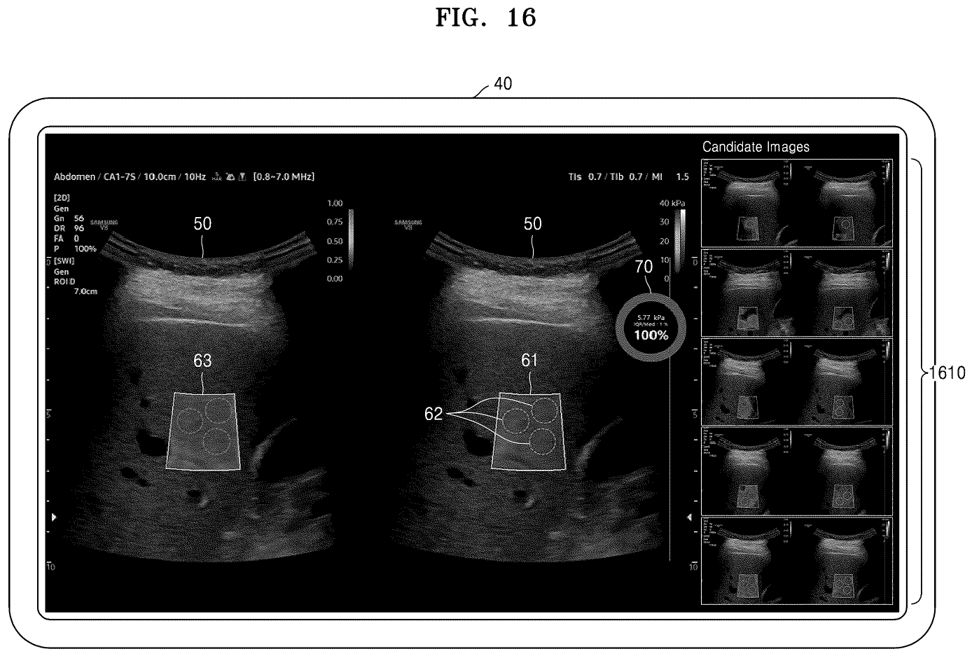

FIG. 16 illustrates a method, performed by the ultrasound imaging apparatus 40, of displaying elasticity images, according to an embodiment.

Referring to FIG. 16, the ultrasound imaging apparatus 40 may display a list of a plurality of elasticity images for which elasticity data is obtained.

As elastography is repeatedly performed on a ROI of an object, the ultrasound imaging apparatus 40 may repeatedly obtain the elasticity images at a time interval. The time interval may be, for example, 1 second or 1.5 seconds, but is not limited thereto.

The ultrasound imaging apparatus 40 may display a list 1610 of elasticity images for which the elasticity values are obtained. As shown in FIG. 16, a B mode image, an elasticity image, and a reliability image corresponding to each elasticity image in the list 1610 of elasticity images may be displayed.

According to an embodiment, the ultrasound imaging apparatus 40 may update the elasticity image to the list 1610 of elasticity images whenever an elasticity image is obtained, and an elasticity value is obtained from the obtained elasticity image. An elasticity image for which an elasticity value has not been obtained may not be updated to the list 1610 of elasticity images.

According to an embodiment, after elastography is completed, the ultrasound imaging apparatus 40 may display the list 1610 of elasticity images for which the elasticity values are obtained.

Whenever an elasticity image 61 is obtained, the ultrasound imaging apparatus 40 may determine an elasticity measurement ROI 62 in the elasticity image 61, and display the determined elasticity measurement ROI 62 in the elasticity image 61 and a reliability image 63.

The ultrasound imaging apparatus 40 may display a position of an elasticity measurement ROI corresponding to each elasticity image on each elasticity image in the list 1610 of elasticity images.

As a user input for scrolling the list 1610 of elasticity images is received, the ultrasound imaging apparatus 40 may display hidden elasticity images.

The machine-readable storage medium may be provided in the form of a non-transitory storage medium. The term ‘non-transitory storage medium’ may mean a tangible device without including a signal, e.g., electromagnetic waves, and may not distinguish between storing data in the storage medium semi-permanently and temporarily. For example, the non-transitory storage medium may include a buffer that temporarily stores data.

In an embodiment, the aforementioned method according to the various embodiments of the disclosure may be provided in a computer program product. The computer program product may be a commercial product that may be traded between a seller and a buyer. The computer program product may be distributed in the form of a machine-readable storage medium (e.g., a CD-ROM) or distributed directly between two user devices (e.g., smart phones) or online (e.g., downloaded or uploaded). In the case of the online distribution, at least part of the computer program product (e.g., a downloadable app) may be at least temporarily stored or arbitrarily created in a storage medium that may be readable to a device such as a server of the manufacturer, a server of the application store, or a relay server.

What is claimed is:

1. An ultrasound imaging apparatus comprising:
an ultrasound transceiver module;
a memory storing instructions; and
at least one processor comprising processing circuitry,
wherein, when the instructions are executed individually or collectively by the at least one processor, the ultrasound imaging apparatus is configured to
repeatedly perform elastography on a region of interest (ROI) through the ultrasound transceiver module, based on receiving a user input for obtaining an elasticity value of the ROI of an object,
identify an elasticity measurement ROI on an elasticity image when the elasticity image of the ROI is obtained through the elastography,
obtain an elasticity value from the identified elasticity measurement ROI,
accumulate the obtained elasticity value as elasticity data,
display information about the number of the accumulated elasticity data together with the obtained elasticity image, and
obtain a next elasticity image through next elastography.

2. The ultrasound imaging apparatus of claim 1, wherein
the at least one processor is configured to execute the instructions to
obtain the next elasticity image through the next elastography on the ROI based on the number of the elasticity data being less than a target number or a volatility indicator of the elasticity data not satisfying a volatility indicator condition, and
display information indicating that the elasticity data has been sufficiently obtained based on the number of the elasticity data being greater than or equal to the target number and the volatility indicator of the elasticity data satisfying the volatility indicator condition.

3. The ultrasound imaging apparatus of claim 1, wherein the at least one processor is configured to execute the instructions to end the elastography based on the number of the elasticity data being greater than or equal to the target number and the volatility indicator of the elasticity data satisfying the volatility indicator condition.

4. The ultrasound imaging apparatus of claim 1, wherein the at least one processor is configured to execute the instructions to determine a representative value of the elasticity data as the elasticity value of the ROI based on the number of the elasticity data being greater than or equal to the target number and the volatility indicator of the elasticity data satisfying the volatility indicator condition.

5. The ultrasound imaging apparatus of claim 3, wherein a representative value of the elasticity data is a median of the elasticity data, the volatility indicator is a ratio between an interquartile range (IQR) value of the elasticity data and the median, and the volatility indicator condition is that the ratio is within a predetermined ratio.

6. The ultrasound imaging apparatus of claim 3, wherein
the at least one processor is configured to execute the instructions to
select elasticity values of the target number from among the elasticity data as the number of elasticity data exceeds the target number,
update the selected elasticity values with the elasticity data, and
identify whether the number of the updated elasticity data is greater than or equal to the target number and whether the volatility indicator of the updated elasticity data satisfies the volatility indicator condition.

7. The ultrasound imaging apparatus of claim 1, wherein the at least one processor is configured to execute the instructions to display information indicating a ratio of the target number to the number of elasticity data in which the elasticity value is accumulated as a progress of measurement of the elasticity value when the elasticity image is obtained.

8. The ultrasound imaging apparatus of claim 1, wherein the at least one processor is configured to execute the instructions to display a volatility indicator of elasticity data in which the elasticity value is accumulated when the elasticity image is obtained.

9. The ultrasound imaging apparatus of claim 1, wherein the at least one processor is configured to execute the instructions to display the identified elasticity measurement ROI on the elasticity image.

10. The ultrasound imaging apparatus of claim 1, wherein the at least one processor is configured to execute the instructions to exclude an elasticity value, which is obtained from a deleted elasticity image, from the elasticity data based on receiving a user input for deleting the elasticity image.

11. A method, performed by an ultrasound imaging apparatus, of providing information about elastography, the method comprising:
repeatedly performing elastography on a region of interest (ROI), based on receiving a user input for obtaining an elasticity value of the ROI of an object;
identifying an elasticity measurement ROI on an elasticity image when the elasticity image of the ROI is obtained through the elastography;
obtain an elasticity value from the identified elasticity measurement ROI;
accumulating the obtained elasticity value as elasticity data;
displaying information about the number of the accumulated elasticity data together with the obtained elasticity image; and
obtaining a next elasticity image through next elastography.

12. The method of claim 11, wherein based on the number of the accumulated elasticity data being less than a target number, the obtaining of the next elasticity image through the next elastography includes obtaining the next elasticity image through the next elastography on the ROI based on the number of the elasticity data being less than the target number or a volatility indicator of the elasticity data not satisfying a volatility indicator condition,
the method further comprising: displaying information indicating that the elasticity data has been sufficiently obtained based on the number of the elasticity data being greater than or equal to the target number and the volatility indicator of the elasticity data satisfying the volatility indicator condition.

13. The method of claim 11, further comprising: ending the elastography based on the number of elasticity data being greater than or equal to the target number and the volatility indicator of the elasticity data satisfying the volatility indicator condition.

14. The method of claim 11, further comprising: determining a representative value of the elasticity data as the elasticity value of the ROI based on the number of the elasticity data being greater than or equal to the target number and the volatility indicator of the elasticity data satisfying the volatility indicator condition.

15. The method of claim 13, wherein a representative value of the elasticity data is a median of the elasticity data, the volatility indicator is a ratio between an interquartile range (IQR) value of the elasticity data and the median, and the volatility indicator condition is that the ratio is within a predetermined ratio.

16. The method of claim 13, further comprising:
selecting elasticity values of the target number from among the elasticity data when the number of elasticity data exceeds the target number;
updating the selected elasticity values with the elasticity data; and
identifying whether the number of the updated elasticity data is greater than or equal to the target number and whether the volatility indicator of the updated elasticity data satisfies the volatility indicator condition.

17. The method of claim 11, further comprising: displaying information indicating a ratio of the target number to the number of elasticity data in which the elasticity value is accumulated as a progress of measurement of the elasticity value when the elasticity image is obtained.

18. The method of claim 11, further comprising: displaying a volatility indicator of elasticity data in which the elasticity value is accumulated as the elasticity image is obtained.

19. The method of claim 11, further comprising: displaying the identified elasticity measurement ROI on the elasticity image.

20. The method of claim 11, further comprising: excluding an elasticity value, which is obtained from a deleted elasticity image, from the elasticity data based on receiving a user input for deleting the elasticity image.
